(19) United States
(12) Patent Application Publication
(10) Pub. No.: US 2019/0367876 A1
(43) Pub. Date: Dec. 5, 2019

(54) METHODS OF TRANSDUCING AND EXPANDING IMMUNE CELLS AND USES THEREOF

(71) Applicant: F1 ONCOLOGY, INC., West Palm Beach, FL (US)

(72) Inventors: Gregory Ian FROST, West Palm Beach, FL (US); James Joseph ONUFFER, West Palm Beach, FL (US)

(73) Assignee: F1 ONCOLOGY, INC., West Palm Beach, FL (US)

(21) Appl. No.: 16/478,377

(22) PCT Filed: Jan. 17, 2018
(86) PCT No.: PCT/US2018/014116

§ 371 Date: Jul. 16, 2019

Related U.S. Application Data
(60) Provisional application No. 62/447,894, filed on Jan. 18, 2017.
(60) Provisional application No. 62/447,913, filed on Jan. 19, 2017.
(60) Provisional application No. 62/467,062, filed on Mar. 3, 2017.

(51) Int. Cl. C12N 5/0783

(52) U.S. Cl. CPC C12N 5/0638; C12N 5/0646; C12N 2500/32; C12N 2510/00; C12N 2501/2307; C12N 2501/51; C12N 2501/515; C12N 2501/2302

(57) ABSTRACT

The present disclosure provides methods for genetically modifying and expanding immune cells ex vivo, especially for use in cell-based adoptive immunotherapy. As such, method embodiments are provided for transducing immune cells (e.g. T cells and/or NK cells) that include a step of activating the cells and genetically modifying the activated cells, for example by transducing the cells with recombinant retroviral particles, such as lentiviral particles. Genetically modified cells produced by these methods are also provided. Such methods are typically performed within a closed system, and in illustrative embodiments within a single chamber of a closed system. The methods typically include expanding the genetically modified immune cells in cell expansion media within the closed system, in illustrative embodiments within the single chamber of the closed system. As such, provided herein in illustrative embodiments, are fed-batch, single-reactor method systems.

### CROSS-REFERENCE TO RELATED APPLICATIONS

This application is claims the benefit of U.S. Provisional Application No. 62/447,894, filed Jan. 18, 2017, U.S. Provisional Application No. 62/447,913, filed Jan. 19, 2017, and U.S. Provisional Application No. 62/467,062, filed Mar. 3, 2017. These applications cited in this paragraph are incorporated by reference herein in their entirety.

### FIELD OF DISCLOSURE

This disclosure relates to methods for transducing and expanding immune cells ex vivo.

### BACKGROUND OF THE DISCLOSURE

In adoptive cell therapy, immune cells isolated from a patient can be genetically modified ex vivo to express synthetic proteins that enable the cells to perform new therapeutic functions after they are subsequently transferred back into the patient. Before the cells are transferred back into the patient, the modified cells are typically expanded ex vivo to provide a sufficient number of cells to perform the therapeutic function. Prior methods of modifying and expanding the immune cells for cell-based adoptive immunotherapy can be difficult, labor-intensive, cost prohibitive, and include multiple steps where contamination can occur. Furthermore, such methods can be limited in their effectiveness to help many people who would benefit from them because they can require special skills and equipment such that their deployment can be limited to only specialized ex vivo manufacturing facilities.

There remains a need for a relatively simple, safe method to isolate immune cells and genetically modify and expand the genetically modified immune cells ex vivo. Such methods would expand the deployment of methods of adoptive cell therapy, such as chimeric antigen receptor technologies (CAR-T), which have resulted in unprecedented curative rates for certain types of cancers and holds promise for many patients who currently are in need of an effective cancer treatment. Such patients often do not have the health or financial means to travel many miles to a specialized ex vivo manufacturing facility to receive a therapy that could be curative for them. Thus, remains a need for methods for performing adoptive cell therapies that are more amenable to widespread deployment because they are simpler and more cost-effective than current methods.

### SUMMARY OF THE DISCLOSURE

Numerous aspects and embodiments are provided herein in the Detailed Description for transducing T cells and/or NK cells, typically ex vivo. As non-limiting examples, illustrative methods provided herein, include a step of activating T cells and/or NK cells and genetically modifying the activated T cells and/or NK cells (for example by transducing the T cells and/or NK cells with recombinant retroviruses or recombinant retroviral particles (typically replication incompetent recombinant retroviral particles and in illustrative embodiments replication incompetent lentiviral particles)) within a closed system, in illustrative embodiments within a single chamber of a closed system, to produce genetically modified T cells and/or NK cells. Typically, such methods further include enriching PBMCs from blood or a blood fraction to isolate PBMCs comprising the T cells and/or NK cells that are activated in the activating step. Furthermore, in illustrative embodiments, such methods typically include after transducing the T cells and/or NK cells, expanding the genetically modified T cells and/or NK cells in cell expansion media within the closed system, in illustrative embodiments within the single chamber of the closed system.

In some embodiments, the T cells and/or NK cells (in illustrative embodiments T cells) can be genetically modified to express a chimeric antigen receptor (CAR), by transducing the T cells and/or NK cells with nucleic acids including the nucleotide sequences encoding the CAR. The T cells and/or NK cells genetically modified according to the methods of the present disclosure can be used in various methods, which are also provided herein, including methods for performing adoptive cell therapy such as CAR therapy, for example CAR therapy against cancer.

Details of aspects and embodiments, such as the non-limiting exemplary methods discussed above, are provided throughout this disclosure. For the sake of clarity, such non-limiting exemplary embodiments provided in this Summary section are not intended to be, and should not be construed to be limiting the scope of the disclosure provided in this entire disclosure.

### BRIEF DESCRIPTION OF THE DRAWINGS

FIG. 1 shows an exemplary protocol for isolating PBMCs, and activating, transducing, expanding, and harvesting T cells and/or NK cells from the isolated PBMCs.

FIG. 2 shows the ex vivo fold expansion, percent transduction, and viability of cells after performing the activation, transduction, and expansion methods provided in Example 1. The bars indicate fold expansion. The line shows percent transduction efficiency. The numbers on top of the bars show the percent viability of each treatment group. Culture base media M1-M4 (see Example 1 for details) for the activation, transduction, and expansion are noted. RetroNectin pretreatment is noted. Cells were activated and transduced in a G-Rex chamber, a plate, or a bag. For expansion, the cells either remained in the G-Rex chamber (Direct G-Rex) or were transferred to G-Rex chambers from the plate (Plate to G-Rex) and bag (Cultilife Bag) as noted.

FIG. 3 shows the percentage of CD3+ cells that are CD4+ or CD8+. Culture base media M1-M4 (see Example 1 for details) for the activation, transduction, and expansion are noted. RetroNectin pretreatment is noted. Cells were activated and transduced in a G-Rex chamber, a plate, or a bag. For expansion, the cells either remained in the G-Rex chamber (Direct G-Rex) or were transferred to G-Rex chambers from the plate (Plate to G-Rex) and bag (Cultilife Bag) as noted.

FIG. 4 shows the expansion fold, percent viability, and the percentage of CD3+eTAG+ cells for cells activated and expanded in the presence or absence of antibodies to CD28 and media with different supplements.

FIG. 5 shows the percentage of CD3+eTAG+ cells for cells activated and expanded in the presence or absence of IL-7 and NAC at Day 0 or Day 2.

FIG. 6 shows the volume of blood and PBMC yield from Subjects 13, 21, and 28 and the percentages of CD3+ cells, CD+CD8+ cells, CD3+CD4+ cells, CD3+CD56+ cells, and CD-CD56+ cells at day 0, before the PBMCs were subjected to an activating step.

FIG. 7 shows the lactate concentration in the media during the expansion of PBMCs from Subjects 13, 21, and 28 that were transduced with a lentivirus particle preparation encoding an Ax1 MRB-CAR or a Ror2 MRB-CAR.

FIG. 8 shows the expansion fold and percent viability of PBMCs from Subjects 13, 21, and 28 that were expanded after transduction with a lentivirus particle preparation encoding an Ax1 MRB-CAR or a Ror2 MRB-CAR.

FIG. 9 shows the harvest day of PBMCs from human subjects 13, 21, and 28 that were expanded after transduction with a lentivirus particle preparation encoding an Ax1 MRB-CAR or a Ror2 MRB-CAR and the percentages of CD3+eTAG+ cells, CD3+ cells, CD3+CD8+ cells, CD3+CD4+ cells, CD3+CD56+ cells, and CD3-CD56+ cells in the harvested cells.

FIG. 10 shows the blood volume and PBMC yield at day 0, before activation, for two samples, A or B, processed separately for 4 subjects (1-4) and the percentages of CD3+, CD3+CD8+ cells, CD3+CD4+ cells, CD3+CD56+ cells, CD3-CD56+ cells, CD14+ monocytes, and CD14+ lymphocytes in the day 0 samples.

FIGS. 11A and 11B show the lactate (11A) and glucose (11B) concentration in the media of samples 1A, 1B, 2A, 2B, 3A, and 4A after activation and transduction, during expansion on days 4, 6, 8, 10, and 12. The identity of the samples is provided in FIG. 10.

FIG. 12 shows the total number of expanded cells harvested (Total Live) after activation, transduction, and expansion, the expansion fold, the cell viability of the harvested cells, and the percentages of CD3+eTAG+ cells, CD3+ cells, CD3+CD8+ cells, CD3+CD4+ cells, CD3+CD56+ cells, and CD3-CD56+ cells in harvested cells for samples 1A, 1B, 2A, 2B, 3A, and 4A. The identity of the samples is provided in FIG. 10.

### DEFINITIONS

The terms “peripheral blood mononuclear cells” or “PBMCs” as used herein refer to any peripheral blood cell having a round nucleus. PBMCs include lymphocytes, such as T cells, B cells, and NK cells, and monocytes.

The term “immune cells” as used herein generally includes white blood cells (leukocytes) which are derived from hematopoietic stem cells (HSC) produced in the bone marrow “Immune cells” includes, e.g., lymphocytes (T cells, B cells, natural killer (NK) (CD3−CD56+) cells) and myeloid-derived cells (neutrophil, eosinophil, basophil, monocyte, macrophage, dendritic cells). “T cells” include all types of immune cells expressing CD3 including T-helper cells (CD4⁺ cells), cytotoxic T-cells (CD8⁺ cells), T-regulatory cells (Treg) and gamma-delta T cells, and NK T cells (CD3+ and CD56+). A skilled artisan will understand T cells and/or NK cells, as used throughout the disclosure, can include only T cells, only NK cells, or both T cells and NK cells. In certain illustrative embodiments and aspects provided herein, T cells are activated and transduced. Furthermore, T cells are provided in certain illustrative composition embodiments and aspects provided herein. A “cytotoxic cell” includes CD8⁺ T cells, natural-killer (NK) cells, NK-T cells, γδT cells, and neutrophils, which are cells capable of mediating cytotoxicity responses.

The term “genetically modified” as used herein includes methods to introduce exogenous nucleic acids into a cell, whether or not the exogenous nucleic acids are integrated into the genome of the cell.

The term “genetically modified cell” as used herein includes cells that contain exogenous nucleic acids whether or not the exogenous nucleic acids are integrated into the genome of the cell.

As used herein, “lymphodepletion” involves methods that reduce the number of lymphocytes in a subject, for example by administration of a lymphodepletion agent, such as monoclonal antibodies or cytotoxic drugs. Lymphodepletion can also be attained by partial body or whole body fractioned radiation therapy. A lymphodepletion agent can be a chemical compound or composition capable of decreasing the number of functional lymphocytes in a mammal when administered to the mammal. One example of such an agent is one or more chemotherapeutic agents. Such agents and dosages are known, and can be selected by a treating physician depending on the subject to be treated. Examples of lymphodepletion agents include, but are not limited to, fludarabine, cyclophosphamide, cladribine, denileukin diftitox, or combinations thereof.

The terms “chimeric antigen receptor” or “CAR” or “CARs” as used herein refer to engineered receptors, which graft an antigen specificity onto cells, for example T cells, NK cells, macrophages, and stem cells. CARs can include at least one antigen-specific targeting region (ASTR), a hinge or stalk domain, a transmembrane domain (TM), one or more co-stimulatory domains (CSDs), and an intracellular activating domain (IAD). In certain embodiments, the CSD is optional. In another embodiment, the CAR is a bispecific CAR, which is specific to two different antigens or epitopes. After the ASTR binds specifically to a target antigen, the IAD activates intracellular signaling. For example, the IAD can redirect T cell specificity and reactivity toward a selected target in a non-MHC-restricted manner, exploiting the antigen-binding properties of antibodies. The non-MHC-restricted antigen recognition gives T cells expressing the CAR the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains.

The terms “polynucleotide” and “nucleic acid”, as used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “antibodies” and “immunoglobulin” as used herein include antibodies or immunoglobulins of any isotype, fragments of antibodies which retain specific binding to antigen, including, but not limited to, Fab, Fab′, Fab′-SH, (Fab′)₂ Fv, scFv, divalent scFv, and Fd fragments, chimeric antibodies, humanized antibodies, single-chain antibodies, and fusion proteins comprising an antigen-specific targeting region of an antibody and a non-antibody protein.

The term “antibody fragments” as used herein include a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fe” fragment, a designation reflecting the ability to crystallize readily. Pepsin treatment yields an F(ab′)₂ fragment that has two antigen combining sites and is still capable of cross-linking antigen.

The terms “single-chain Fv,” “scFv,” or “sFv” as used herein refer to antibody fragments that include the V_{H} and V_{L} domains of antibody, wherein these domains are present in a single polypeptide chain. In some embodiments, the Fv polypeptide further includes a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, N.Y., pp. 269-315 (1994).

The term “affinity” as used herein refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” as used herein refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁻⁷ M, e.g., binding with an affinity of 10⁻⁶ M, 10⁻⁵ M, 10⁻⁴ M, etc.

The term “region” as used herein is any segment of a polypeptide or polynucleotide.

The term “domain” as used herein is a region of a polypeptide or polynucleotide with a functional and/or structural property.

The terms “stalk” or “stalk domain” as used herein refer to a flexible polypeptide connector region providing structural flexibility and spacing to flanking polypeptide regions and can consist of natural or synthetic polypeptides. A stalk can be derived from a hinge or hinge region of an immunoglobulin (e.g., IgG1) that is generally defined as stretching from Glu216 to Pro230 of human IgG1 (Burton (1985) Molec. Immunol., 22:161-206). Hinge regions of other IgG isotypes may be aligned with the IgG1 sequence by placing the first and last cysteine residues forming inter-heavy chain disulfide (S—S) bonds in the same positions. The stalk may be of natural occurrence or non-natural occurrence, including but not limited to an altered hinge region, as disclosed in U.S. Pat. No. 5,677,425. The stalk can include a complete hinge region derived from an antibody of any class or subclass. The stalk can also include regions derived from CD8, CD28, or other receptors that provide a similar function in providing flexibility and spacing to flanking regions.

The term “hinge region” as used herein refers to a flexible polypeptide connector region (also referred to herein as “hinge” or “spacer”) providing structural flexibility and spacing to flanking polypeptide regions and can consist of natural or synthetic polypeptides. A “hinge region” derived from an immunoglobulin (e.g., IgG1) is generally defined as stretching from Glu216 to Pro230 of human IgG1 (Burton (1985) Molec. Immunol., 22:161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425. The hinge region can include a complete hinge region derived from an antibody of a different class or subclass from that of the CH1 domain. The term “hinge region” can also include regions derived from CD8, CD28, or other receptors that provide a similar function in providing flexibility and spacing to flanking regions.

The term “isolated polypeptide” as used herein is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

The term “stem cell” as used herein generally includes pluripotent or multipotent stem cells. “Stem cells” includes, e.g., embryonic stem cells (ES); mesenchymal stem cells (MSC); induced-pluripotent stem cells (iPS); and committed progenitor cells (hematopoietic stem cells (HSC); bone marrow derived cells, etc.).

The terms “treatment,” “treating,” and the like, as used herein refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e g , in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” as used interchangeably herein, refer to a mammal, including, but not limited to, humans, murines (e.g., rats, mice), lagomorphs (e.g., rabbits), non-human primates, canines, felines, and ungulates (e.g., equines, bovines, ovines, porcines, caprines). “Donors” as referred to herein are human subjects that donated blood.

The terms “effective amount”, “therapeutically effective amount”, or “efficacious amount” as used herein refer to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

The terms “physiological”, “normal”, or “normal physiological” conditions as used herein are conditions such as, but not limited to, temperature, pH, osmotic pressure, osmolality, oxidative stress and electrolyte concentration, as well as other parameters, that would be considered within a normal range at the site of administration, or at the tissue or organ at the site of action, to a subject.

It is to be understood that the present disclosure and the aspects and embodiments provided herein, are not limited to particular examples disclosed, as such may, of course, vary. It is also to be understood that the terminology used herein is for the purpose of disclosing particular examples and embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. When multiple low and multiple high values for ranges are given, a skilled artisan will recognize that a selected range will include a low value that is less than the high value.

All headings in this specification are for the convenience of the reader and are not limiting.

Unless defined otherwise, all technical and scientific terms used herein have the same meaning as commonly understood by one of ordinary skill in the art to which this invention belongs. Although any methods and materials similar or equivalent to those disclosed herein can also be used in the practice or testing of the present invention, the preferred methods and materials are now disclosed.

It must be noted that as used herein and in the appended claims, the singular forms “a,” “an,” and “the” include plural referents unless the context clearly dictates otherwise. Thus, for example, reference to “a chimeric antigen receptor” includes a plurality of such chimeric antigen receptors and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### DETAILED DESCRIPTION

Provided herein are methods for manipulating and typically expanding, immune cells, especially T cells and/or NK cells, that require less sample handling and manipulation than prior methods, thereby providing methods that are simpler and more streamlined Thus, these methods reduce the likelihood of human error and microbial contamination. Furthermore, these methods help to facilitate effective performance of such methods by more laboratories, thus expanding the access to cells that are processed using the methods. Illustrative aspects and embodiments disclosed herein provide methods for a single reactor, fed-batch process for the enrichment, activation, transduction, and expansion of T cells and/or NK cells in a closed system. The instant methods, in illustrative aspects, advantageously generate large numbers of transduced T cells and/or NK cells in a short time from a small volume of blood.

Illustrative methods provided herein, which are typically ex vivo methods, include a step of activating T cells and/or NK cells and transducing the activated T cells and/or NK cells with recombinant retroviruses or recombinant retroviral particles (typically replication incompetent recombinant retroviral particles and in illustrative embodiments replication incompetent recombinant lentiviral particles) within a closed system, typically within a single chamber (also referred to as a reactor, vessel, container, compartment, or receptacle) of a closed system, to produce genetically modified T cells and/or NK cells. The chamber can be flexible or rigid. In illustrative embodiments, the chamber can be rigid. Typically, such methods further include enriching PBMCs to isolate PBMCs comprising the T cells and/or NK cells that are used in the activating step. Furthermore, in illustrative embodiments, such methods typically include after transducing the T cells and/or NK cells, expanding the genetically modified T cells and/or NK cells in cell expansion media within the closed system, typically within the single chamber of the closed system. In illustrative embodiments of methods provided herein, T cells are activated, transduced and typically expanded. Such T cells in illustrative embodiments are genetically modified with a chimeric antigen receptor (CAR).

Illustrative embodiments provided herein include no wash step between the activation and the transduction and no wash step between the transduction and the expansion. Thus, in these illustrative embodiments, the activation, transduction, and expansion are performed within the same chamber of the closed system, and without washing or removing the T cells and/or NK cells from the chamber from the start of the activation through expansion. Thus, an activating agent such as anti-CD3 antibodies, that are included in the activation step, are typically present and detectable during the transduction and expansion steps. Furthermore, illustrative embodiments provided herein, which can include isolating PBMCs from as little as 50 ml of collected blood, result in at least 10 times as many genetically modified T cells and/or NK cells after expansion than present in the isolated PBMCs.

In one aspect provided herein is a method for transducing T cells and/or NK cells from isolated blood, including: a) isolating peripheral blood mononuclear cells (PBMCs) including T cells and/or NK cells from isolated blood; b) activating T cells and/or NK cells of the isolated PBMCs under effective conditions within a closed system and without enriching T cells and/or NK cells from other PBMCs, including an effective amount of anti-CD3 antibody in solution; and c) transducing the activated T cells and/or NK cells with replication incompetent recombinant retroviral particles under effective conditions, thereby producing genetically modified T cells and/or NK cells, wherein the activating and transducing are performed within the same closed system without washing the cells between the activating and transducing. In further embodiments, the method can further include expanding the genetically modified T cells and/or NK cells in cell expansion media. In illustrative embodiments, the activating, transducing, and expanding is performed within the same chamber of the same closed system.

In illustrative embodiments, no more than 20% or 10% of the cell media is removed during the enriching, activating, transducing, or expanding. In such illustrative embodiments, media is removed only in small samples, such as 2 ml or 1 ml or less samples, to assess the progress of a step, such as the expanding, and/or to assess the number, health, compositions, and/or status of cells during the method. In illustrative embodiments, no washing is performed between activation, transduction, and expansion, and therefore anti-CD3 antibody, typically added as part of the activation, is present in the media during the expanding. In illustrative embodiments, N-acetyl cysteine (NAC) is absent during the transducing, but present in the media during the expanding. In some embodiments, the media can be supplemented with cytokines during the transducing, expanding, and harvesting. In the methods disclosed herein, the media is typically further supplemented with cytokines during the expanding. In illustrative embodiments, the media can be supplemented with IL-2 and optionally IL-7 during the expanding.

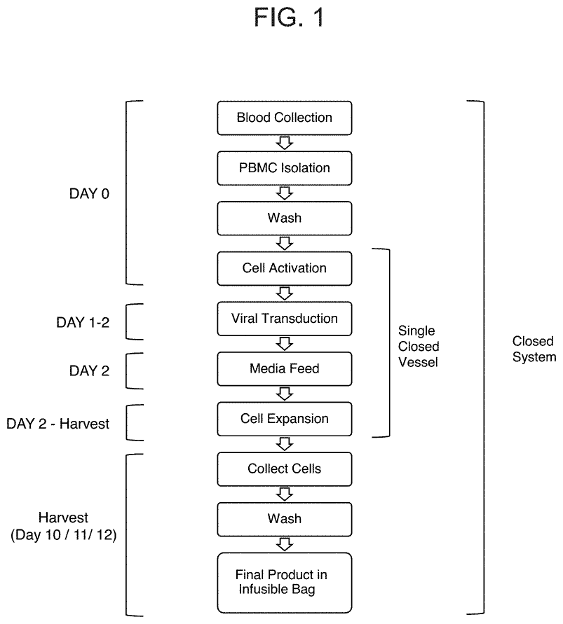

A non-limiting embodiment is presented in FIG. 1. On day 0, blood is collected from a subject and PBMCs are enriched to isolate PBMCs comprising T cells and/or NK cells from the blood and washed within a closed system. The PBMCs are then typically transferred to a chamber within the closed system to activate, transduce, and expand the T cells and/or NK cells. Activating agents are then added to the chamber to begin activation of the isolated PBMCs. On day 1, replication incompetent recombinant retroviral particles are added to the chamber to begin transducing the activated cells. On day 2, the cells are fed with cell expansion media to begin cell expansion. The cell expansion media typically includes a base media that is present during the activating and the transducing steps. In illustrative embodiments, the media for cell expansion can be supplemented with N-acetyl-cysteine (NAC), which in illustrative embodiments is absent in the activating step and the transducing step. The cell expansion media can be supplemented, for example with 10 mM NAC. In further illustrative embodiments, the base media in the expansion step, as well as the activation and the transduction steps, can be supplemented with cytokines, for example, IL-2 and optionally IL-7. After expansion, the cells can be harvested when the lactate concentration in the cell expansion media reaches or exceeds a predetermined concentration, for example 10 mM or in illustrative embodiments, 20 mM, or at day 12 if the predetermined concentration of lactate has not been reached. The cells are harvested by collecting, washing, and either transferring them to an infusible bag or cryopreserving them in a vial or, preferably, a cryo bag.

As indicated above, in some embodiments, the media is supplemented with cytokines during the cell expansion, for example, 100 IU/ml IL-2 and optionally 10 ng/ml IL-7. In some embodiments, the media can be further supplemented with IL-2 and optionally IL-7 every 12, 24, or 48 hours during the expanding. In illustrative embodiments, the media is supplemented during expanding with IL-2 and optionally IL-7 on days 4, 6, and 8. In some embodiments, during expanding the media can be supplemented with IL-2 at a final concentration between 50, 60, 70, 80, 90, 100, 110, or 120 IU/ml on the low end of the range and 60, 70, 80, 90, 100, 110, 120, 130, 140, or 150 IU/ml on the high end of the range. In illustrative embodiments, the steps from activating to expanding the cells can be performed in a single closed chamber in the closed system. In fact in illustrative embodiments, the method from blood collection through harvesting, is performed in a closed system, such that the cells are not exposed to the environment at any point during the method. In illustrative embodiments, no more than 20% of the media is removed during the entire method and thus the activating agents, such as anti-CD3 antibody and/or anti-CD28 antibody, are present throughout the transducing and expanding steps.

In illustrative embodiments, the steps of activating T cells and/or NK cells, transducing the T cells and/or NK cells with replication incompetent recombinant retroviral particles to produce genetically modified T cells and/or NK cells, expanding the genetically modified T cells and/or NK cells, and harvesting the expanded T cells and/or NK cells are performed in a closed system. A closed system is a cell processing system that is generally closed or fully closed to an environment, such as an environment within a room or even the environment within a hood, outside of the conduits such as tubes, and chambers, of the system in which cells are processed, cultured, and/or transported. One of the greatest risks to safety and regulatory control in the cell processing procedure is the risk of contamination through frequent exposure to the environment as is found in traditional open cell culture systems. To mitigate this risk, particularly in the absence of antibiotics, some commercial processes have been developed that focus on the use of disposable (single-use) equipment. However, even with their use under aseptic conditions, there is always a risk of contamination from the opening of flasks to sample or add additional growth media. To overcome this problem, methods provided herein, which are typically ex vivo methods, are typically performed within a closed-system. Such a process is designed and can be operated such that the product is not exposed to the outside environment. Material transfer occurs via sterile connections, such as sterile tubing and sterile welded connections. Air for gas exchange can occur via a gas permeable membrane, via 0.2 μm filter to prevent environmental exposure. In addition, using the methods provided herein, T cells and/or NK cells can be activated, transduced, and expanded without washing the T cells and/or NK cells between or during these steps. Furthermore, activating agents, such as anti-CD3 antibody, in illustrative embodiments, is in solution, and thus the substrate, such as a bead, that anti-CD3 antibody is often attached to, does not have to be removed in such illustrative embodiments. In further illustrative embodiments, the methods are performed on T cells, for example to provide genetically modified T cells.

Such closed system methods can be performed with commercially available devices. Different closed system devices can be used at different steps within a method and the cells can be transferred between these devices using tubing and connections such as welded, luer, spike, or clave ports to prevent exposure of the cells or media to the environment. For example, blood can be collected into an IV bag or syringe and transferred to a Sepax 2 device (Biosafe) for PBMC enrichment and isolation. The isolated PBMCs can be transferred to a chamber of a G-Rex device for activation, transduction, and expansion. Finally, the cells can be harvested and collected into another bag using a Sepax 2 device. The methods can be carried out in any device or combination of devices adapted for closed system T cell and/or NK cell production. Non-limiting examples of such devices include G-Rex devices (Wilson Wolf), GatheRex (Wilson Wolf), Sepax 2 (Biosafe), WAVE Bioreactors (General Electric), a CultiLife Cell Culture bag (Takara), a PermaLife bag (OriGen), CliniMACS Prodigy (Miltenyi Biotec), and VueLife bags (Saint-Gobain). In illustrative embodiments, the activating, transducing, and expanding can be performed in the same chamber or vessel in the closed system. For example, in illustrative embodiments, the chamber can be a chamber of a G-Rex device and PBMCs can be transferred to the chamber of the G-Rex device after they are enriched and isolated, and can remain in the same chamber of the G-Rex device until harvesting.

Closed systems have typically included steps to coat some or all of the surfaces of the devices which come into contact with the cells. For example, the surfaces can be coated with recombinant fibronectin or fragments of fibronectin, such as RetroNectin (Takara), which, not to be limited by theory, can promote transduction of T cells and/or NK cells by replication incompetent recombinant retroviral particles. Before the cells can be introduced into the coated devices, washes must be performed. The steps of coating and washing the devices introduce more risks for contamination. Therefore, in any of embodiments disclosed herein, T cells and/or NK cells can advantageously be introduced into the closed system devices without coating the surfaces. In some embodiments, the methods can be performed in the absence of recombinant fibronectin or RetroNectin.

### Blood Collection

Blood containing PBMCs can be collected or obtained from a subject by any suitable method known in the art. For example, the blood can be collected by venipuncture or any other blood collection method by which a sample of blood and/or PBMCs is collected. In some embodiments, the volume of blood collected is typically between 50 ml and 250 ml, for example, between 75 ml and 125 ml, or between 90 ml and 120 ml, or between 95 and 110 ml. In some embodiments, the volume of blood collected can be between 25, 30, 35, 40, 45, 50, 55, 60, 65, 70, 75, 80, 85, 90, 95, 100, 110, 120, 130, 140, 150, 175, 200, 225, 250, 275, 300, 350, 400, 450, 500, 600, 700, 800, or 900 ml on the low end of the range and 30, 35, 40, 45, 50, 55, 60, 65, 70, 75, 80, 85, 90, 95, 100, 110, 120, 130, 140, 150, 175, 200, 225, 250, 275, 300, 350, 400, 450, 500, 600, 700, 800, or 900 ml or 1 L on the high end of the range. In the methods disclosed herein, large numbers of genetically modified T cells and/or NK cells can be generated in a short amount of time, such as 10-14 days, from a small volume of blood, such as between 80 ml and 100 ml. In some embodiments, PBMCs can be obtained by apheresis as discussed below. However, during apheresis, larger volumes of blood are typically taken and processed than if the blood is collected. In some embodiments, the volume of blood taken and processed during apheresis can be between 0.5, 0.6, 0.7, 0.75, 0.8, 0.9, 1, 1.25, or 1.5 total blood volumes of a subject on the low end of the range and 0.6, 0.7, 0.75, 0.8, 0.9, 1, 1.25, 1.5 1.75, 2, 2.25, or 2.5 total blood volumes of a subject on the high end of the range. The total blood volume of a human typically ranges from 4.5 to 6 L and thus much more blood is taken and processed during apheresis than if the blood is collected and then the PBMCs are isolated, as in illustrative embodiments herein.

### Enrichment of PBMCs

In methods for adoptive cell therapy and any method provided herein that include transducing T cells and/or NK cells ex vivo, peripheral blood mononuclear cells (PBMCs) including T cells and/or NK cells, are isolated away from other components of a blood sample in an enrichment step. Such enrichment can be performed, for example, by forming a buffy coat from a blood sample using known methods. Then PBMCs can be enriched by collecting the buffy coat and further enriching PBMCs from other blood components using known methods. In some embodiments, contaminating red blood cells in the sample taken for quantitation can be lysed using known methods before counting. Enrichment of PBMCs from other blood components and blood cells can be performed, for example, using apheresis, and/or density gradient centrifugation. In some embodiments, neutrophils are removed before T cells and/or NK cells are processed, contacted with replication incompetent recombinant retroviral particles, transduced, or transfected. With reference to a subject to be treated, the cells can be allogeneic and/or autologous.

In illustrative embodiments, the PBMCs are enriched and isolated using a Sepax or Sepax 2 cell processing system (BioSafe). In some embodiments, the PBMCs are enriched and isolated using a CliniMACS Prodigy cell processor (Miltenyi Biotec). In any of the embodiments disclosed herein, density gradient centrifugation can be performed, for example using a Sepax cell processing system. In some embodiments, Ficoll-Paque (GE Healthcare) can be used. In some embodiments, an automated apheresis separator is used which takes blood from the subject, passes the blood through an apparatus that sorts out a particular cell type (such as, for example, PBMCs), and returns the remainder back into the subject. Density gradient centrifugation can be performed after apheresis. In some embodiments, the PBMCs can be enriched and isolated using a leukoreduction filter device. In some embodiments, magnetic bead activated cell sorting is then used for purifying a specific cell population from PBMCs, such as, for example, T cells and/or NK cells, according to a cellular phenotype (i.e. positive selection). In some embodiments, monocytes and/or macrophages can be removed from the PBMCs using methods known in the art. For example, monocytes and/or macrophages can be removed using magnetic bead activated cell sorting (i.e. negative selection) or by allowing the PBMCs to grow on tissue-culture treated surfaces such that the monocytes and/or macrophages adhere, and then transferring the supernatant to a new container. However, in certain illustrative embodiments, methods provided herein, and in particular activating steps, are performed on PBMCs without enriching for other cell types such as T cell and/or NK cells. In some embodiments, PBMCs can be cryopreserved according to methods known in the art. However, in illustrative embodiments, PBMCs are activated without first cryopreserving.

During the PBMC enrichment process, one or more washes can be performed as is known in the art, before the enriched PBMCs are isolated and then activated. For example, the washes can be performed on the Sepax 2 system used for enriching the PBMCs. The wash solution can any solution suitable for washing blood and/or PBMCs. In some embodiments, the washing solution can be saline supplemented with human serum albumin (HSA), human AB+ serum, serum derived from the subject, or a synthetic sera replacement. In some embodiments, the HSA, human AB+ serum, serum derived from the subject, or a synthetic sera replacement can be present in the washing solution at a final concentration of between 0.25%, 0.5%, 1%, 1.5%, 2%, 3%, 4%, 5%, 6%, 7%, 8%, or 9% on the low end of the range and 1%, 2%, 3%, 4%, 5%, 6%, 7%, 8%, 9%, or 10% on the high end of the range. In illustrative embodiments, the HSA, human AB+serum, serum derived from the subject, and/or a synthetic sera replacement can be present in the washing solution at a final concentration of between 0.5%, 1%, or 1.5% on the low end of the range and 1%, 2%, 3%, 4%, or 5% on the high end of the range. In further illustrative embodiments, the washing solution can be saline supplemented with HSA at a final concentration of between 0.5%, 1%, or 1.5% on the low end of the range and 1%, 2%, 3%, 4%, or 5% on the high end of the range.

In any of the embodiments disclosed herein, the number of PBMCs isolated can be between 1×10⁶, 2.5×10⁶, 5×10⁶, or 1×10⁷ PBMCs on the low end of the range and 2.5×10⁶, 5×10⁶, 1×10⁷, 2.5×10⁷, 5×10⁷, 1×10⁸, 2.5×10⁸, 5×10⁸, or 1×10⁹ PBMCs on the high end of the range. In illustrative embodiments, the number of PBMCs isolated can be between 5×10⁶, 1×10⁷, 2.5×10⁷, 5×10⁷ PBMCs on the low end of the range and 1×10⁷, 2.5×10⁷, 5×10⁷, or 1×10⁸ PBMCs on the high end of the range. According to methods known in the art, the isolated PBMCs can be resuspended in any suitable base culture medium used for culturing T cells and/or NK cells ex vivo, including base media and supplements, including cytokines, as disclosed in further detail within the cell expansion section herein below.

In any of the embodiments disclosed herein, the PBMCs can be resuspended in between 50, 60, 70, 80, 90, 100, 125, 150, 175, or 200 ml media on the low end of the range and 60, 70, 80, 90, 100, 125, 150, 175, 200, 250, 300, 400, or 500 ml on the high end of the range. In some embodiments, the PBMCs can be resuspended in at least 50, 60, 70, 80, 90, 100, 125, 150, 175, or 200 ml media. In illustrative embodiments, the PBMCs can be resuspended in at most 50, 60, 70, 80, 90, 100, 125, 150, 175, or 200 ml media.

### Activation of PBMCs

In any of the embodiments disclosed herein, the methods typically include a step of activating or stimulating the isolated PBMCs with one or more activating agents in an activation reaction mixture to generate activated T cells and/or NK cells. In some embodiments, the isolated PBMCs are transferred to a chamber within another device in the closed system, such as a G-Rex device, before the activating. Activating can be performed with either freshly isolated PBMCs or previously cryopreserved PBMCs. In the event that cryopreserved cells are used, the cells may be thawed using developed protocols prior to use. In some embodiments, the activation can be performed without centrifugation.

The number of isolated PMBCs to activate can be adjusted such that after expansion, a sufficient number of genetically modified T cells and/or NK cells are harvested for introducing, reintroducing, or transferring back into a patient. In some embodiments, the number of PBMCs to activate are between 1×10⁶, 2.5×10⁶, 5×10⁶, or 1×10⁷ PBMCs on the low end of the range and 2.5×10⁶, 5×10⁶, 1×10⁷, 2.5×10⁷, 5×10⁷, 1×10⁸, 2.5×10⁸, 5×10⁸, or 1×10⁹ PBMCs on the high end of the range. In illustrative embodiments, the number of PBMCs to activate are between 5×10⁶, 1×10⁷, 2.5×10⁷, 5×10⁷ PBMCs on the low end of the range and 1×10⁷, 2.5×10⁷, 5×10⁷, or 1×10⁸ PBMCs on the high end of the range. In some embodiments, media, such as base cell culture media, can be added to the isolated PBMCs to adjust the cell density of the PBMCs to between 1×10³, 2.5×10³, 5×10³, 1×10⁴, 2.5×10⁴, 5×10⁴, 1×10⁵, 2.5×10⁵, or 5×10⁵ PBMCs/ml on the low end of the range and 2.5×10³, 5×10³, 1×10⁴, 2.5×10⁴, 5×10⁴, 1×10⁵, 2.5×10⁵, 5×10⁵, 1×10⁶, 2.5×10⁶, 5×10⁶, or 1×10⁷ PBMCs/ml on the high end of the range. In illustrative embodiments, media is added to the isolated PBMCs to adjust the cell density of the PBMCs to between 5×10³, 1×10⁴, 2.5×10⁴, or 5×10⁴, 1×10⁵ PBMCs/ml on the low end of the range and 1×10⁴, 2.5×10⁴, 5×10⁴, 1×10⁵, 2.5×10⁵, or 5×10⁵ PBMCs/ml on the high end of the range. In some embodiments, where less than a threshold number of PBMCs are isolated (for example, fewer than 1×10⁶, 5×10⁶, 1×10⁷, 5×10⁷ or 1×10⁸ PBMCs), the total volume of cell culture media added is lowered to achieve PBMC cell densities within the desired recited range,

Media is typically present during the activating, such as those known in the art for culturing of T cells and/or NK cells ex vivo, including base media and supplements including cytokines, as disclosed in further detail within the cell expansion section herein below.

Any combination of one or more activating agents can be used to produce activated T cells and/or NK cells. The one or more activating agents can be added to the media in the chamber of the closed system without exposing the PBMCs to the environment. A reaction mixture is typically formed within the chamber of the closed system to perform the activating. In some embodiments, the reaction mixture can be formed by adding one or more activating agents to the media. In any of the embodiments disclosed herein, the one or more activating agents are used in effective amounts such that activated T cells and/or NK cells are produced. In some embodiments, the activating agent can be an antibody or a functional fragment thereof that targets or binds to a T-cell stimulatory or co-stimulatory molecule, or any other suitable mitogen (e.g., tetradecanoyl phorbol acetate (TPA), phytohaemagglutinin (PHA), concanavalin A (conA), lipopolysaccharide (LPS), pokeweed mitogen (PWM)) or natural ligand to a T-cell stimulatory or co-stimulatory molecule. Some prior methods have supplemented the activation reaction mixture with aminobisphosphonates. However, in illustrative embodiments herein, no aminobisphosphonates (natural or synthetic) are present in the activation reaction mixture.

Various antibodies and functional fragments thereof are known in the art to activate or stimulate T cells and/or NK cells. In some embodiments, anti-CD2, anti-CD3, and/or anti-CD28 can be added to the media. In illustrative embodiments, anti-CD3 and anti-CD28 antibodies can be added to the media. In further illustrative embodiments, anti-CD3 antibody alone can be added to the media. In some embodiments, the one or more antibodies or functional fragments thereof can be immobilized on a solid surface, such as a bead. In any of the embodiments disclosed herein, anti-CD28 can be CD80 or CD86 or any functional fragment thereof that retains the ability to bind CD28. However, the use of immobilized antibodies or functional fragments thereof typically necessitates their removal at some point during the methods. In illustrative embodiments, the one or more antibodies or functional fragments thereof can advantageously be in solution. Not to be limited by theory, in some embodiments, the one or more antibodies or functional fragments thereof in solution can be bound by antigen-presenting cells, for example other PBMCs, present during the activating. In some embodiments, the one or more antibodies or functional fragments thereof are not attached to or immobilized on a synthetic solid support, such as a bead.

In some embodiments, anti-CD3 antibody, IL-2, and in some sub-embodiments, anti-CD28 or a polypeptide capable of binding to CD28 such as CD80 or CD86, and/or IL-7 can be added to the media during the activating. In illustrative embodiments, anti-CD3 antibody, IL-2, and IL-7 can be added to the media during the activating. As part of a continuous, fed-batch process where media is not removed, one or more activating agents, such as anti-CD3 antibody, can remain in the media during the transducing, expanding, and harvesting.

After addition of the one or more activating agents, the T cells and/or NK cells can be incubated at between 23 and 39° C., and in some illustrative embodiments at 37° C. In some embodiments, the activation reaction can be carried out at 37-39° C. The T cells and/or NK cells can be incubated with the one or more activating agents for between 8, 9, 10, 11 12, 13, 14, 15, 16, 17, 18, 19, 20, 21, 22, 23, or 24 hours on the low end of the range and 12, 13, 14, 15, 16, 17, 18, 19, 20, 21, 22, 23, 24, 27, 30, 36, 40 or 48 hours on the high end of the range. In illustrative embodiments, the T cells and/or NK cells can be incubated with the one or more activating agents for between 15, 16, 17, 18, 19, 20, 21, 22, 23, or 24 on the low end of the range and 18, 19, 20, 21, 22, 23, 24, 27, 30, or 36 on the high end of the range, such as between 18 and 30 hours.

Transduction of T Cells and/or NK Cells

Methods are provided herein, for genetically modifying T cells and/or NK cells, as well as T cells and/or NK cells that are produced by such methods. In some embodiments of such methods and compositions disclosed herein, T cells and/or NK cells are contacted ex vivo with replication incompetent recombinant retroviral particles to genetically modify the T cells and/or NK cells. Not to be limited by theory, during the period of contact the replication incompetent recombinant retroviral particles bind to T cells and/or NK cells at which point the retroviral and host cell membranes start to fuse. Then, through the process of transduction, genetic material from the replication incompetent recombinant retroviral particles enters the T cells and/or NK cells and typically is incorporated into the host cell DNA. Accordingly, such methods include genetically modifying T cells and/or NK cells by transduction. Methods are known in the art for transducing T cells and/or NK cells ex vivo with replication incompetent recombinant retroviral particles, such as replication incompetent recombinant lentiviral particles. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. In some embodiments, the T cells and/or NK cells can be contacted with replication incompetent recombinant retroviral particles. In illustrative embodiments, the T cells and/or NK cells can be contacted with replication incompetent recombinant lentiviral particles. In some embodiments, the transduction can be performed without centrifugation.

The transduction reaction of the methods provided herein can be performed in a closed system. Typically, transduction is performed in the same chamber of the closed system as the activating is performed without removing any of the media. For example, blood cells, such as PBMCs enriched and isolated from the collected blood sample, can be activated in the G-Rex system and then contacted with replication incompetent recombinant retroviral particles in the same G-Rex system. In illustrative embodiments, blood cells are separated, isolated, and/or purified away from granulocytes, including neutrophils, which are typically not present during the contacting step, i.e., the transduction reaction and activated according to the methods discussed elsewhere herein. The replication incompetent recombinant retroviral particles, which in further illustrative embodiments can be replication incompetent recombinant lentiviral particles, are introduced into the closed system that contains the activated PBMCs, in illustrative sub-embodiments the same chamber of the closed system in which the activating is performed, to form a transduction reaction mixture. In some embodiments, the replication incompetent recombinant retroviral particles are added to the reaction mixture formed during the activating step. Media is typically present during the transduction, such as those known in the art for culturing of T cells and/or NK cells ex vivo, including base media and supplements including cytokines, as disclosed in further detail within the cell expansion section herein below.

The transduction reaction, which in some embodiments begins when the replication incompetent recombinant retroviral particles are added, can be incubated at between 23 and 39° C., and in some illustrative embodiments at 37° C. In some embodiments, the transduction reaction can be carried out at 37-39° C. for faster fusion/transduction. The transduction reaction can be incubated for between 8, 9, 10, 11 12, 13, 14, 15, 16, 17, 18, 19, 20, 21, 22, 23, or 24 hours on the low end of the range and 12, 13, 14, 15, 16, 17, 18, 19, 20, 21, 22, 23, 24, 27, 30, 36, 40 or 48 hours on the high end of the range. In illustrative embodiments, the transduction reaction can be incubated for between 15, 16, 17, 18, 19, 20, 21, 22, 23, or 24 on the low end of the range and 18, 19, 20, 21, 22, 23, 24, 27, 30, or 36 on the high end of the range, such as between 18 and 30 hours.

In an illustrative embodiment, blood is collected from a subject into a blood bag and the blood bag is attached to a cell processing system, such as a Sepax 2 cell processing system. PBMCs enriched and isolated using the cell processing system are collected into a bag in media, transferred to a G-Rex system, activated, contacted with replication incompetent recombinant retroviral particles in conditions sufficient to transduce T cells and/or NK cells, and incubated. During the transduction, genetically modified T cells and/or NK cells are produced. After incubation, media is added to the G-Rex chamber containing the mixture of PBMCs and replication incompetent recombinant retroviral particles, to expand the genetically modified T cells and/or NK cells, until a specific cell density or lactate concentration is reached or until a certain number of days has been reached. In some embodiments, the cells are collected and attached to a cell processing system and the T cells and/or NK cells are washed. The washed T cells and/or NK cells are collected into a bag and reinfused into the subject.

T cells and/or NK cells can be transduced with different ratios of replication incompetent recombinant retroviral or lentiviral particles to cells, referred to as the multiplicity of infection (MOI). In some embodiments, the T cells and/or NK cells can be transduced using a MOI between 0.25, 0.5, 1, 2, 3, 4, 5, 6, 7, 8, 9, or 10 on the low end of the range and 0.5, 1, 2, 3, 4, 5, 6, 7, 8, 9, 10, 11, 12, 13, 14, or 15 on the high end of the range. In illustrative embodiments, the T cells and/or NK cells can be transduced using a multiplicity of infection (MOI) between 1, 2, 3, 4, or 5 on the low end of the range and 3, 4, 5, 6, 7, 8, 9, or 10 on the high end of the range.

In some embodiments of the methods and compositions disclosed herein, between 5% and 90% of the total T cells and/or NK cells isolated from the blood can be transduced. In some embodiments, the percent of T cells and/or NK cells that are transduced can be between 5%, 10%, 15%, 20%, 25%, 30%, 35%, 40%, 45%, 50%, 55%, or 60% on the low end of the range, and 50%, 55%, 60%, 65%, 70%, 75%, 80%, 85%, or 90% on the high end of the range. In some embodiments, the percent of T cells and/or NK cells that are transduced can be at least 5%, 10%, 15%, 20%, 25%, 30%, 35%, 40%, 45%, 50%, 55%, or 60%.

Expansion of Transduced T Cells and/or NK Cells

In illustrative embodiments of the methods disclosed herein, transduced T cells and/or NK cells can be expanded before harvesting. In illustrative embodiments, the current disclosure provides methods for a single reactor, fed-batch process within a closed system. In fed-batch processes, fresh media and/or supplements are added to the closed system. However, no media or cells are removed throughout the method beyond small samples, such as 1 to 2 ml or less samples, used to analyze the media and/or the cells. This advantageously reduces the risks of contamination, as well as providing a simpler method with reduced labor and reagent costs. Previous methods for transducing T cells and/or NK cells have included one or more washes during or after one or more steps between activating the T cells and/or NK cells and before the start of (e.g. before an initial cell collection step of) harvesting the genetically modified T cells and/or NK cells, with each wash increasing the risk of contamination as well as expense. In illustrative embodiments of methods provided herein, no washes are performed during any steps between activating the T cells and/or NK cells and before the start of (e.g. before an initial cell collection step of) harvesting the genetically modified T cells and/or NK cells.

In some embodiments, the expanding can be performed without centrifugation. In illustrative embodiments, the activating, transducing, and expanding can be performed without centrifugation. In any of the embodiments disclosed herein, cell expansion media can be added to the transduced cells to perform the expansion. In some embodiments, at least 100, 150, 200, 250, 300, 400, 500, 600, 700, 750, 800, or 900 ml or 1, 1.5, 2, 3, 4, 5, 6, 7, 8, 9, 10, 15, 20, 25, or 30 L of cell expansion media can be added to the transduced cells to perform the expansion. In some embodiments, the transduced cells can be diluted by a factor of at least 2, 3, 4, 5, 6, 7, 8, 9, 10, 11, 12, 13, 14, 15, or 20 fold to perform the expansion. In some embodiments, media can be added to the reaction mixture formed during the activating. In illustrative embodiments, the media added to the isolated PBMCs remains or is not removed until the transduced cells are expanded and harvested. In illustrative embodiments, none of the media is removed during or between the activating, transducing, or expanding. In some embodiments, less than 20%, 10%, 5%, 4%, 3%, 2%, 1%, or 0.1%, of the media is removed during or between the activating, transducing, or expanding. In some embodiments, the only media removed during the method between the activating and the start of the harvesting, is for a sample of 10 ml, 5 ml, 2.5 ml, 2 ml, or 1 ml or less, to count or otherwise assess the status of the cells being processed, or to analyze media composition, such as lactate concentration.

Expanding the T cells and/or NK cells can occur in the same chamber in which the T cells and/or NK cells were transduced, which in illustrative embodiments is the same chamber in which the T cells and/or NK cells were activated. In illustrative embodiments, the chamber can be a chamber of a G-Rex device or a CliniMACS Prodigy device. In further illustrative embodiments, no media is removed from the chamber during or between the activating, transducing, and expanding. In some embodiments, the supplements added to the media during previous steps are present in the media during the expanding. For example, in some embodiments, one or more activating agents such as anti-CD3 antibody and/or anti-CD28 antibody can be present in the media during expanding. Thus, the activating agent can be present at a concentration greater than 1/1,000^{th}, 1/500^{th}, 1/250^{th}, 1/100^{th}, 1/50^{th}, 1/25^{th}, 1/20^{th}, or 1/10^{th} the concentration of the activating agent present during the activating. In illustrative embodiments, the measurement of the concentration of such activating agent is taken at any point during the activating step and at the beginning of the expanding step. In some embodiments, anti-CD3 antibody can be present during the expanding, and in illustrative embodiments at the beginning of the expanding, at a concentration greater than 1/1,000^{th}, 1/500^{th}, 1/250^{th}, 1/100^{th}, 1/50^{th}, 1/25^{th}, 1/20^{th}, or 1/10^{th} the concentration of anti-CD3 antibody present during the activating. In some embodiments, anti-CD28 antibody can be present in the media at the begging of expanding or during expanding at a concentration greater than 1/100^{th}, 1/50^{th}, 1/25^{th}, 1/20^{th}, or 1/10^{th} the concentration of anti-CD28 antibody present during the activating. In some embodiments, anti-CD3 antibody and/or anti-CD28 antibody can be present in the media at the beginning of, or during expanding at a concentration equal to or 5%, 10%, 15%, 20%, or 25% less than the initial concentration in the activating divided by the dilution factor between the volume of base media or an activation reaction mixture of the activating step and the volume of media added at the beginning of the expanding. Not to be limited by theory, it is believed in some embodiments, that some of the activation agents will be removed from the media by being taken up by some fraction of PBMCs, such as monocytes. Thus, the concentration of the one or more activating agents in the cell expansion media, for example, can be less than the dilution factor of the activation step reaction mixture volume to the expansion media volume. However, the amount of activating agents during expansion is expected to be detectable and much higher relatively, than the amount of trace if any activating agents present during expansion in methods that include one or more wash steps between activation and expansion.

Previous methods have included one or more washes between activating the PBMCs and expanding the transduced T cells and/or NK cells, for example, between the activating and transducing and/or between the transducing and expanding and/or during any of these steps. In illustrative embodiments, no washes are performed during or between activating, transducing, and expanding. In illustrative embodiments, no cells are removed from the closed system from activating through expanding, and in further illustrative embodiments, through harvesting.

Typically, ex vivo culture immune cell media, especially T cell and/or NK cell, and most especially T cell media is present throughout the cell activation, cell transduction, and cell expansion steps of methods provided herein. In illustrative embodiments, the same base media is present throughout the cell activation, cell transduction, and cell expansion. Furthermore, in certain illustrative embodiments, other than NAC, the same supplements, including media supplements such as serum replacement and cytokines, are present during the cell activation, cell transduction and cell expansion. For example, in illustrative embodiments, IL-2 and optionally IL-7 is present throughout the process from activating the PBMCs until harvesting the genetically modified T cells and/or NK cells. In certain illustrative embodiments, NAC is present during the expansion, but absent during the transduction, and optionally present during the activation. In certain illustrative embodiments, supplemental NAC, in addition to any NAC that might be present in a base media, is present during the expansion, but absent during the transduction, and optionally present during the activation. For cell expansion, in illustrative embodiments, media, also referred to herein as cell expansion media, is added to the chamber in the closed system after transducing. Other than the presence of activating agents for the activation step, the presence of expression vectors such as replication incompetent retroviral particles for the transduction step, and the presence of supplemental NAC, in addition to any NAC present in the cell expansion media, for expansion, the same composition of the cell expansion media can be present in the activation, the transduction, and the expansion.

In illustrative embodiments, the cell expansion media is serum-free media. In illustrative embodiments, the cell expansion media contains no natural sera. It will be understood that natural sera is sera obtained directly from an organism. In further illustrative embodiments, the cell expansion media can include a serum replacement, as are known in the art. The media can include base media such as commercially available media for ex vivo T cell and/or NK cell expansion such as, as non-limiting examples, X-VIVO™ 15 Chemically Defined, Serum-free Hematopoietic Cell Medium (Lonza) (2018 catalog numbers BE02-060F, BE02-00Q, BE-02-061Q, 04-744Q, or 04-418Q), ImmunoCult™-XF T Cell Expansion Medium (STEMCELL Technologies) (2018 catalog number 10981), PRIME-XV® T Cell Expansion XSFM (Irvine Scientific) (2018 catalog number 91141), AIM V® Medium CTS™ (Therapeutic Grade) (Thermo Fisher Scientific (Referred to herein as “Thermo Fisher”), or CTS™ Optimizer™ media (Thermo Fisher) (2018 catalog numbers A10221-01 (basal media (bottle)), and A10484-02 (supplement), A10221-03 (basal media (bag)), A1048501 (basal media and supplement kit (bottle)) and, A1048503 (basal media and supplement kit (bag)). Such media can be a chemically defined, serum-free formulation manufactured in compliance with cGMP. The media can be xeno-free and complete. In some embodiments, the base media has been cleared by regulatory agencies for use in ex vivo cell processing, such as an FDA 510(k) cleared device. In some embodiments, the media is the basal media with or without the supplied T cell expansion supplement of 2018 catalog number A1048501 (CTS™ OpTmizer™ T Cell Expansion SFM, bottle format) or A1048503 (CTS™ OpTmizer™ T Cell Expansion SFM, bag format) both available from Thermo Fisher (Waltham, Mass.). It will be understood herein where it is recited that the cell expansion media comprises the composition of the basal media with media supplement of a catalog number such as A1048501 or A1048503 that includes a media supplement, that the recited composition is intended to mean the composition of the media with the added supplement. Typically, the manufacture of the media and supplement provides instructions for volumes of supplement to be added.

In some embodiments, the media is further supplemented with supplements in addition to or instead of supplements provided in commercial kits with the media. In some embodiments, the media can be supplemented with HSA, human AB+ serum, serum derived from the subject, and/or serum replacement. In illustrative embodiments, the media can be supplemented with a serum replacement, such as a xeno-free formulation that does not contain bovine or other non-human, animal-derived components, such as CTS™ Serum Replacement (Thermo Fisher) (2018 catalog number A2596102). In some embodiments, the media can be supplemented with HSA, human AB+ serum, serum derived from the subject and/or serum replacement at a final concentration between 0.25%, 0.5%, 1%, 1.5%, 2%, 3%, 4%, 5%, 6%, 7%, 8%, or 9% on the low end of the range and 1%, 2%, 3%, 4%, 5%, 6%, 7%, 8%, 9%, or 10% on the high end of the range. In illustrative embodiments, the media can be supplemented with HSA, human AB+ serum, serum derived from the subject and/or serum replacement at a final concentration between 0.25%, 0.5%, 1%, or 1.5% on the low end of the range and 1%, 1.5%, 2%, 3%, 4%, or 5% on the high end of the range. In some embodiments, the basal media is supplemented with OpTmizer™ CTS™ T-Cell Expansion Supplement (Available in 2018 catalog numbers AF10484-02, Thermo Fisher) and/or L-glutamine or L-alanyl-L-glutamine, a dipeptide substitute for L-glutamine (See CTS™ GlutaMAX™-I Supplement (2018 catalog number A1286001, Thermo Fisher)).

In some embodiments, the media can be supplemented with cytokines, such as IL-2 and/or IL-7, before and/or during expanding. The cytokine such as IL-2 and/or IL-7 are/is typically not from the same subject that is the initial T cells that were transfected or transduced and being expanded, and is typically purchased from a commercial source. Cytokines are known in the art to be beneficial for the growth of T cells and/or NK cells, including isolated, wildtype, or recombinant forms of interleukin 1 (IL-1), interleukin 2, (IL-2), interleukin 4 (IL-4), interleukin 5 (IL-5), interleukin 7 (IL-7), interleukin 15 (IL-15), and tumor necrosis factor α (TNFα). Any of these cytokines or any combination of these cytokines can be added to the media during the expanding. In illustrative embodiments, the concentration of IL-2 is lower than is typical in the art. In some embodiments, the concentration of IL-2 in the media can be between 5, 10, 15, 20, 25, 30, 35, 40, 45, 50, 60, 70, 80, 90, 100, 125, 150, 200, or 250 IU/ml on the low end of the range and 10, 15, 20, 25, 30, 35, 40, 45, 50, 60, 70, 80, 90, 100, 125, 150, 200, 250, or 300, 400, 500, 600, 700, 800, 900, or 1,000 IU/ml on the high end of the range. In some embodiments, the concentration of IL-2 in the media can be less than 10, 15, 20, 25, 30, 35, 40, 45, 50, 60, 70, 80, 90, 100, 125, 150, 200, 250, or 300 IU/ml. In some illustrative embodiments, the concentration of IL-2 in the media can be between 75, 100, 125, or 150 IU/ml on the low end of the range and 200, 250, or 275 IU/ml on the high end of the range. In some embodiments, the concentration of IL-7 in the media can be between 0, 0.1, 0.5, 0.75, 1, 2, 3, 4, 5, 6, 7, 8, 9, 10, 15, 20, 25, 30, 35, 40, or 45 ng/ml on the low end of the range and 0.5, 0.75, 1, 2, 3, 4, 5, 6, 7, 8, 9, 10, 15, 20, 25, 30, 35, 40, 45, or 50 ng/ml on the high end of the range. In some embodiments, the concentration of IL-7 in the media can be less than 0.1, 0.5, 0.75, 1, 2, 3, 4, 5, 6, 7, 8, 9, 10, 15, 20, 25, 30, 35, 40, 45 or 50 ng/ml, or IL-7 can be absent from the cell expansion media, and optionally any media formed during the entire method. In some embodiments, IL-2 and optionally IL-7 can be present from activating the PBMCs until harvesting the genetically modified T cells and/or NK cells. In some embodiments, the media can be supplemented with IL-2 and optionally IL-7 multiple times during expanding. In certain illustrative embodiments, the media can be supplemented with IL-2. For example, every 12, 24, 36, or 48 hours. In illustrative embodiments, the media can be supplemented with IL-2 and optionally IL-7, 24 hours after expansion is started and every 48 hours thereafter until harvesting.

In previous methods for activating, transducing, and expanding T cells and/or NK cells, the media has either contained the same concentration of N-acetyl-cysteine (NAC) for all steps of the method or NAC has been omitted entirely. However, in the methods disclosed herein supplemental NAC, in addition to any NAC present in a basal media, was found to be inhibitory during the transducing but beneficial during the expanding. Thus, in illustrative embodiments disclosed herein, supplemental NAC is omitted from the media during activating, and transducing T cells and/or NK cells, although NAC may be present in the basal media, for example at a concentration of NAC present in CTS™ Optimizer™ media. Then, supplemental NAC is added to the cell expansion media. In some embodiments, at least 1, 2, 3, 4, 5, 6, 7, 8, 9, 10, 11, 12, 13, 14, 15, 16, 17, 18, 19, or 20 mM supplemental NAC can be added to the media. Thus, in illustrative embodiments, the concentration of NAC in the cell expansion media is greater than the concentration of NAC in the media during the activating or transducing. In some embodiments, supplemental NAC can be added to the cell expansion media such that the concentration of NAC present in the cell expansion media is at least 1, 2, 3, 4, 5, 6, 7, 8, 9, 10, 11, 12, 13, 14, 15, 16, 17, 18, 19, or 20 mM higher than the concentration of NAC present in the media during the transducing. In certain illustrative embodiments, the cell expansion media comprises a concentration of NAC that is between 5 mM and 20 mM or between 5 mM and 15 mM, or between 7.5 mM and 12.5 mM or 9 mM and 11 mM or 10 mM greater than the concentration of NAC present during the transduction reaction. In certain illustrative embodiments, the cell expansion media comprises a concentration of NAC that is between 5 mM and 20 mM or between 5 mM and 15 mM, or between 7.5 mM and 12.5 mM or 9 mM and 11 mM or 10 mM greater than the concentration of NAC present in CTS™ Optimizer™ media. In these embodiments, NAC can be absent from the transduction reaction mixture and/or the activation transduction mixture, or present in the transduction reaction mixture and/or the activation reaction mixture at a concentration less than or equal to the concentration of NAC in CTSTM OptimizerTM media.

In certain embodiments, NAC is added to the cell expansion media during the expanding in methods where the PBMCs are enriched and isolated from subjects who are not healthy, for example subjects afflicted by cancer. Not to be limited by theory, it is believed that NAC, and especially supplemental NAC in addition to any NAC in base media used for a transduction reaction, would be especially beneficial for T cells from subjects who are not healthy

During the expanding, the number of cells, for example NK cells, or NK cells and T cells, or in illustrative embodiments T cells, can be increased by at least 2, 3, 4, 5, 6, 7, 8, 9, 10, 15, 20, 25, 30, 35, 40, 45, 50, 60, 70, 75, 80, 90, 100, 110, 120, 125, or 130-fold over (i.e. there are 2×, 3×, 4×, 5×, 6×, 7×, 8×, 9×, 10×, 15×, 20×, 25×, 30×, 35×, 40×, 45×, 50×, 60×, 70×, 75×, 80×, 90×, 100×, 110×, 120×, 125×, or 130× as many as) the initial number of isolated PBMCs, or T cells and NK cells, or T cells present at the beginning of the expanding or present during the activating or transducing. In illustrative embodiments, the number of cells, viable cells, PBMCs, or T cells and NK cells, or T cells, can be increased by between 2.5, 5, 6, 7, 8, 9, 10, 15, and 20-fold on the low end of the range, and 25, 30, 40, 50, 60, 70, 75, 80, 90, 100, 110, 120, 125, or 130-fold on the high end of the range, or between 2 and 10-fold, or 2 and 75-fold, or 5 and 75-fold, or 10 and 60-fold, or 20 and 50-fold, 2 and 130 fold, 40 and 135-fold, 50 and 135-fold, 50 and 125-fold, or 50 and 150-fold over the initial number of isolated PBMCs or viable PBMCs, or the number of PBMCs present during the activating, or the number of PBMCs present after the transducing, or the number of T cells and NK cells, or T cells present at the beginning of the expanding. In further embodiments, the number of cells, viable cells, PBMCs, T cells and/or NK cells can be increased by at least 5, 6, 7, 8, 9, 10, 15, 20, 25, 30, 40, 50, 70, 75, 80, 90, 100, 110, 120, 125, or 130-fold over the initial number of isolated PBMCs or viable PBMCs, or the number of PBMCs, T cells and/or NK cells present for the activating, or for the transducing, or at the beginning of the expanding and the concentration of IL-2 in the media during the expanding can be less than 10, 15, 20, 25, 30, 35, 40, 45, 50, 60, 70, 80, 90, 100, 125, 150, 200, 250, or 300 IU/ml or between 50 and 275 IU/ml, or between 150 and 250 IU/ml. In some embodiments, the number of T cells and/or NK cells can be increased by at least 10, 15, 20, 25, 30, 35, 40, 50, 60, 70, 80, 90, 100, 125, 150, 200, 250, 300, 400, 500, or 1,000 fold over the number of T cells and/or NK cells present after isolating PBMCs. In some embodiments, the T cells and/or NK cells can undergo at least 1, 2, 3, 4, 5, 6, 7, 8, 9, 10, 11, 12, 13, 14, 15, 16, 17, 18, 19, or 20 cell divisions during the expanding. In illustrative embodiments, the T cells and/or NK cells can undergo at least 4, 5, 6, 7, 8, 9, or 10, cell divisions during the expanding.

Cell expanding can be performed for a certain number days. In some embodiments, expanding can be performed for 4, 5, 6, 7, 8, 9, 10, 11, 12, 13, 14, 15, 16, 17, 18, 19, 20, or 21 days. In some embodiments, expanding can be performed for between 4, 5, 6, 7, or 8 days on the low end of the range and 9, 10, 11, 12, 13, 14, 15, 16, 17, 18, 19, 20, or 21 days on the high end of the range. In certain illustrative embodiments, expanding is performed for between 6 and 12 days, or between 8 and 10 days.

### Cell Harvesting

In any of the methods disclosed herein, after expanding, the transduced T cells and/or NK cells can be harvested. In some embodiments, the transduced T cells and/or NK cells can be concentrated or collected during harvesting using methods known in the art. In some embodiments, the harvesting can be performed in the same chamber of the closed system. In embodiments where the cells are expanded in a G-Rex, the concentrating can include using a GatheRex machine and removing the supernatant in the G-Rex from the precipitate that includes the T cells and/or NK cells.

In some embodiments, the T cells and/or NK cells can be washed one or more times during the harvesting using any suitable wash solution known in the art. In some embodiments, the wash solution can include 5% dextrose in normal saline. The wash solution can be supplemented with HSA, human AB+ serum, serum derived from the subject and/or a synthetic sera replacement. In some embodiments, HSA, human AB+ serum, serum derived from the subject. and/or synthetic sera replacement can be added to a final concentration between 0.25%, 0.5%, 1%, 1.5%, 2%, 3%, 4%, 5%, 6%, 7%, 8%, or 9% on the low end of the range and 1%, 2%, 3%, 4%, 5%, 6%, 7%, 8%, 9%, or 10% on the high end of the range. In illustrative embodiments, the T cells and/or NK cells can be washed with 5% dextrose in normal saline supplemented with HSA at a final concentration between 0.25%, 0.5%, 1%, or 1.5% HSA on the low end of the range and 1%, 1.5%, 2%, 3%, 4%, or 5% HSA on the high end of the range. In some embodiments, the wash solution can be supplemented with sodium bicarbonate (NaHCO₃) to adjust the pH of the wash solution to physiological pH, or about pH 7.4.

In some embodiments, the T cells and/or NK cells can be transferred during harvesting to another chamber in the closed system before washing the cells one or more times. In some embodiments, the T cells and/or NK cells can be washed using a Sepax 2 system.

At the end of the harvesting, the T cells and/or NK cells can be resuspended in any suitable media known in the art. In some embodiments, the cells can be resuspended in media that includes 5% dextrose in normal saline. In some embodiments, the media can be supplemented with sodium bicarbonate (NaHCO₃) to adjust the pH of the wash solution to physiological pH. The use of sodium bicarbonate (NaHCO₃) in media is well-known in the art and can be added to a final concentration between 1, 2.5, 5, 10, 15, 20, 25, 30, 35, 40, 45, or 50 g/L on the low end of the range and 2.5, 5, 10, 15, 20, 25, 30, 35, 40, 45, 50, 60, 70, 80, 90, or 100 g/L on the high end of the range. In further illustrative embodiments, NaHCO₃ can be added to the final resuspension media to a final concentration between 1, 2.5, 5, 10, or 15 g/L on the low end of the range and 2.5, 5, 10, 15, 20, 25, 30, 35, 40, 45, or 50 g/L on the high end of the range. For example, NaHCO₃ can be added to the final resuspension media to a final concentration of about 20 g/L. In illustrative embodiments, NaHCO₃ is not added to the media while activating, transducing, or expanding the T cells and/or NK cells.

The T cells and/or NK cells can be harvested when a predetermined concentration of a metabolite is reached in the media, such as lactate, and/or after a specified period of time, and/or when the T cells and/or NK cells reach a certain density. In some embodiments, harvesting can be performed based on the lactate concentration or until a specified day and cells are harvested regardless of the lactate concentration.

In any of the embodiments disclosed herein, harvesting of the expanded T cells and/or NK cells can be performed based on an expansion a completion criterion or completion criteria. In some embodiments, the expansion completion criterion can be, or include, lactate concentration, extent of cell expansion, cell density, or a number of days in expansion.

In any of the embodiments disclosed herein, harvesting of the expanded T cells and/or NK cells can be performed based on lactate concentration in the media. For example, in some embodiments, harvesting can be performed when the lactate concentration in the media is between 10, 11, 12, 13, 14, 15, 16, 17, 18, 19, 20, 21, 22, 23, 24, or 25 mM on the low end of the range and 15, 16, 17, 18, 19, 20, 21, 22, 23, 24, 25, 26, 27, 28, 29 or 30 mM on the high end of the range. In illustrative embodiments, the harvesting can be performed when the lactate concentration in the media is between 15 and 25 mM, between 17.5 and 22.5 mM, or between 19 and 21 mM. In some embodiments, harvesting can be performed when the lactate concentration in the media is at least 10, 11, 12, 13, 14, 15, 16, 17, 18, 19, 20, 21, 22, 23, 24, 25, 26, 27, 28, 29 or 30 mM. In illustrative embodiments, harvesting can be performed when the lactate concentration in the media is at least 20 mM. In some embodiments, harvesting can be performed 7, 8, 9, 10, 11, 12, 13, 14, 15, 16, 17, 18, 19, 20, or 21 days after the blood was collected if the lactate concentration in the media has not reached a predetermined concentration.

Cell harvesting can also be performed a certain number days after the blood was collected. In some embodiments, harvesting can be performed 7, 8, 9, 10, 11, 12, 13, 14, 15, 16, 17, 18, 19, 20, or 21 days after the blood was collected. In some embodiments, harvesting can be performed between 7, 8, 9, 10, 11, 12, 13, or 14 days after blood was collected on the low end of the range and 10, 11, 12, 13, 14, 15, 16, 17, 18, 19, 20, or 21 days after blood was collected on the high end of the range. In some embodiments, harvesting can be performed 6, 7, or 8 days after expansion begins on the low end of the range and 8, 9, 10, 11, 12, 13, 14, 15, 16, 17, 18, 19, 20, or 21 days after expansion begins on the high end of the range.

In some embodiments, cell harvesting is performed when the T cells and/or NK cells have been expanded at least 2, 3, 4, 5, 6, 7, 8, 9, 10, 11, 12, 13, 14, 15, 16, 17, 18, 19, 20, or 25-fold, In some illustrative embodiments cell harvesting is performed when the T cells and/or NK cells have been expanded at least 5 fold, at least 10 fold, or at least 20 fold. This expansion is typically measured by counting the cells, or viable cells, harvested after expansion versus the total number of PBMCs, or viable PBMCs, that were activated. In some embodiments where all isolated cells for a subject are placed in an activation reaction mixture, the expansion can be measured by comparing the total number of PBMCs, or viable PBMCs, isolated versus the number of cells, or viable cells, harvested. In other embodiments, expansion is measured by counting the T cells and/or NK cells in the isolated PBMCs or an activation reaction versus the total number of cells at the time of harvest, or in illustrative embodiments, by counting the number of T cells, or NK cells, or NK cells and T cells at the time of harvest.

Cell harvesting can also be performed when the T cells and/or NK cells reach a specified cell density in the media. In some embodiments, harvesting can be performed when the cell density is between 1×10⁵, 2.5×10⁵, 5×10⁵, 1×10⁶, 2.5×10⁶, 5×10⁶, 1×10⁷, 2.5×10⁷, or 5×10⁷ cells/ml on the low end of the range and 2.5×10⁵, 5×10⁵, 1×10⁶, 2.5×10⁶, 5×10⁶, 1×10⁷, 2.5×10⁷, 5×10⁷, 1×10⁸, 2.5×10⁸, 5×10⁸, or 1×10⁹ cells/ml on the high end of the range. In illustrative embodiments, harvesting can be performed when the cell density is between 5×10⁵, 1×10⁶, 2.5×10⁶, 5×10⁶, or 1×10⁷ cells/ml on the low end of the range and 2.5×10⁶, 5×10⁶, 1×10⁷, 2.5×10⁷, or 5×10⁷ cells/ml on the high end of the range. In some embodiments, harvesting can be performed when the cell density is at least 1×10⁵, 2.5×10⁵, 5×10⁵, 1×10⁶, 2.5×10⁶, 5×10⁶, 1×10⁷, 2.5×10⁷, 5×10⁷, 1×10⁸, 2.5×10⁸, 5×10⁸, or 1×10⁹ cells/ml.

The number of T cells and/or NK cells harvested can be at least 2, 3, 4, 5, 6, 7, 8, 9, 10 11, 12, 13, 14, 15, 16, 17 18, 19, or 20 times more than the initial number of T cells and/or NK cells isolated in the PBMCs. In illustrative embodiments, the number of T cells and/or NK cells harvested can be at least 5, 6, 7, 8, 9, 10 11, 12, 13, 14, or 15 times more than the initial number of T cells and/or NK cells isolated in the PBMCs.

In some embodiments, the number of T cells and/or NK cells harvested can be at least 1×10⁷, 2.5×10⁷, 5×10⁷, 1×10⁸, 2.5×10⁸, 5×10⁸, 1×10⁹, 2.5×10⁹, 5×10⁹, 1×10¹⁰, 2.5×10¹⁰, 5×10¹⁰, 1×10¹¹, 2.5×10¹¹, 5×10¹¹, 1×10¹², 2.5×10¹², 5×10¹², 1×10¹³, 2.5×10¹³, 5×10¹³, 1×10¹⁴, 2.5×10¹⁴, 5×10¹⁴, or 1×10¹⁵ T cells and/or NK cells. In illustrative embodiments, the number of T cells and/or NK cells harvested can be at least 1×10⁸, 2.5×10⁸, 5×10⁸, 1×10⁹, 2.5×10⁹, 5×10⁹, or 1×10¹⁰ T cells and/or NK cells.

The harvested cells can include different percentages of T cells and NK cells. In some embodiments, the harvested cells can include at least 50%, 55%, 60%, 65%, 70%, 75%, 80%, 85%, 90%, 95%, or 100% T cells and/or NK cells. In illustrative embodiments, the harvested cells can include at least 50%, 55%, 60%, 65%, 70%, 75%, 80%, 85%, 90%, 95%, or 100% T cells.

In some embodiments, the harvested cells can be introduced, introduced back, reintroduced, infused, or reinfused into a subject. In some embodiments, harvested cells can be cryopreserved as described below before reintroduction into a subject. In illustrative embodiments, harvested cells are introduced, introduced back, reintroduced, infused, or reinfused into a subject without first cryopreserving the cells. The subject is typically the same subject the blood was collected from.

Throughout this disclosure, a transduced T cell and/or NK cell includes progeny of the transduced cells that retain at least one of the nucleic acids that are incorporated into the cell during the ex vivo transduction. In methods herein that recite “reintroducing” a transduced cell, it will be understood that such a cell is typically not in a transduced state when it is collected from the blood of a subject.

### Cell Introduction/Reintroduction

In certain embodiments of the methods disclosed herein, the harvested T cells and/or NK cells can be introduced, introduced back, reintroduced, infused, or reinfused in a subject, in illustrative embodiments for a therapeutic effect. The number of T cells and/or NK cells to be reintroduced can be a predetermined dose, which can be a therapeutically effective dose as provided below. in some embodiments, the predetermined dose can depend on the CAR that is expressed on the cells (e.g., the affinity and density of the antigen-specific targeting region expressed on the transduced T cell and/or NK cell), the type of target cell, the nature of the disease or pathological condition being treated, or a combination of both. In some embodiments, the predetermined dose of harvested cells can be based on the mass of a subject, for example, cells per kilogram of the subject (cells/kg). In any of the embodiments disclosed herein, the number of T cells and/or NK cells to be introduced, reintroduced, or transferred back into a subject can be between 1×10³, 2.5×10³, 5×10³, 1×10⁴, 2.5×10⁴, 5×10⁴, 1×10⁵, 2.5×10⁵, 5×10⁵, 1×10⁶, 2.5×10⁶, 5×10⁶, or 1×10⁷ cells/kg on the low end of the range and 5×10⁴, 1×10⁵, 2.5×10⁵, 5×10⁵, 1×10⁶, 2.5×10⁶, 5×10⁶, 1×10⁷, 2.5×10⁷, 5×10⁷, or 1×10⁸ cells/kg on the high end of the range. In illustrative embodiments, the number of T cells and/or NK cells to be reinfused into a subject can be between 1×10⁴, 2.5×10⁴, 5×10⁴, or 1×10⁵ cells/kg on the low end of the range and 2.5×10⁴, 5×10⁴, 1×10⁵, 2.5×10⁵, 5×10⁵, or 1×10⁶ cells/kg on the high end of the range. In some embodiments, the number of T cells and/or NK cells to be reinfused into a subject can be between than 5×10⁵, 1×10⁶, 2.5×10⁶, 5×10⁶, 1×10⁷, 2.5×10⁷, 5×10⁷, or 1×10⁸ cells on the low end of the range and 2.5×10⁶, 5×10⁶, 1×10⁷, 2.5×10⁷, 5×10⁷, 1×10⁸, 2.5×10⁸, 5×10⁸, or 1×10⁹ cells on the high end of the range.

A subject in any of the aspects disclosed herein can be, for example, an animal, a mammal, and in illustrative embodiments, a human. In some embodiments, the subject can be healthy. In other embodiments, the subject can be other than healthy. In some embodiments, the subject can have or be afflicted with a disease. In illustrative embodiments, the disease can be cancer. A variety of subjects are suitable for treatment with a method of treating cancer. Suitable subjects include any individual, e.g., a human or non-human animal who has cancer, who has been diagnosed with cancer, who is at risk for developing cancer, who has had cancer and is at risk for recurrence of the cancer, who has been treated with an agent for the cancer and failed to respond to such treatment, or who has been treated with an agent for the cancer but relapsed after initial response to such treatment.

Subjects suitable for treatment with an immunomodulatory method include individuals who have an autoimmune disorder; individuals who are organ or tissue transplant recipients; and the like; individuals who are immunocompromised; and individuals who are infected with a pathogen.

### Cell Cryopreservation

In some embodiments, the harvested cells produced by the methods described herein can be cryopreserved at a predetermined dose for use at a later time. Methods and reagents for cryopreserving cells are well-known in the art. Cryopreservation can include one or more washes and/or a step of concentrating the T cells and/or NK cells with a diluent solution, which in illustrative embodiments, is a cryopreservation solution. In some embodiments, the diluent solution can be normal saline, 0.9% saline, PlasmaLyte (PL), 5% dextrose/0.45% NaCl saline solution (D5), human serum albumin (HSA), or a combination thereof. In some embodiments. HSA can be added to the washed and concentrated cells for improved cell viability and cell recovery after thawing. In some embodiments, the washing solution can be normal saline and washed and concentrated cells can be supplemented with HSA, for example 1%, 2%, 3%, 4%, 5%, 6%, 7%, 8%, 9%, or 10% HSA. The method can also include a step of forming a cryopreservation mixture, which includes the T cells and/or NK cells in the diluent solution and a suitable cryopreservative solutionAn some embodiments, the cryopreservative solution can be any suitable cryopreservative solution including, but not limited to, CryoStor10 (BioLife Solution), mixed with the diluent solution of T cells and/or NK cells at a ratio of 1:1 or 2:1. In some embodiments, the cryopreservative solution can include at least 1%, 2%, 3%, 4%, 5%, 6%, 7%, 8%, 9%, or 10% DMSO. In some illustrative embodiments the cryopreservative includes between 15% and 12.5% DMSO or between 5% and 10% DMSO. In some embodiments, HSA can be added to a final concentration in the cryopreservative solution of between 1%, 2%, 3%, 4%, or 5% HSA on the low end of the range and 5%, 6%, 7 8%, 9 10%, 11%, 12%, 13%, 14%, or 15% HSA on the high end of the range. In some embodiments, the method can include a step of freezing the cryopreservation mixture. In one aspect, the cryopreservation mixture is frozen in a controlled rate freezer using a defined freeze cycle at any of the predetermined doses discussed above. The method can include a step of storing the cryopreservation mixture in vapor phase liquid nitrogen or liquid nitrogen.

In some embodiments, the predetermined dose can be a therapeutically effective dose, which can be any therapeutically effective dose as provided below. The predetermined dose can depend on the CAR that is expressed on the cells (e.g., the affinity and density of the cell surface receptors expressed on the cell), the type of target cell, the nature of the disease or pathological condition being treated, or a combination of both. In some embodiments, the predetermined dose of harvested cells can be based on the mass of a subject, for example, cells per kilogram of the subject (cells/kg). In some embodiments, the predetermined dose of harvested cells expressing a CAR can be between 1×10⁵, 2.5×10⁵, 5×10⁵, 7.5×10⁵, 1×10⁶, 2×10⁶, 3×10⁶, 4×10⁶, 5×10⁶, 6×10⁶, 7×10⁶, 8×10⁶, 9×10⁶, or 1×10⁷ cells/kg on the low end of the range and 2.5×10⁵, 5×10⁵, 7.5×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, or 1×10⁸ cells/kg on the high end of the range. In some embodiments, the predetermined dose of harvested cells can be at least 1×10⁵, 2.5×10⁵, 5×10⁵, 7.5×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, and 1×10⁸ cells/kg.

In any of the embodiments disclosed herein, the harvested cells can be cryopreserved in about 0.5-200 ml of cryopreservation media. In some embodiments, the harvested cells can be cryopreserved in about 0.5 ml, about 1 ml, about 5 ml, about 10 ml, about 20 ml, about 30 ml, about 40 ml, about 50 ml, about 60 ml, about 70 ml, about 80 ml, about 90 ml, or about 100 ml of cryopreservation media. In some embodiments, the harvested cells can be cryopreserved in between 0.25, 0.5, 0.75, 1, 2, 5, 10, 15, 20, 25, 30, 40, 50, 75, or 100 ml cryopreservation media on the low end of the range and 0.5, 0.75, 1, 2, 5, 10, 15, 20, 25, 30, 40, 50, 75, 100, 125, 150, 175, or 200 ml cryopreservation media on the high end of the range. In some embodiments, the genetically modified T and/or NK cells can be formulated in CS250 cryostorage bags (OriGen Biomedical, Austin, Tex.) to achieve the target dose in a solution containing saline (e.g. 0.9% saline) optionally plus HSA (e.g. 5% HAS) or sera replacement, then diluted 1:1 with Cryostor 10 (BioLife Solutions, Bothell, Wash.).

Methods to thaw cryopreserved T cells and/or NK cells are known in the art. For example, cryopreserved cells can be quickly thawed at 37° C. in a water bath, bead bath, or a commercial controlled thaw rate device and transferred to a container with pre-warmed media. In some embodiments, the cells can be washed with media to remove the cryopreservative solution. In some embodiments, the cells can be allowed to recover for one or more days. In some embodiments, the cells can be used immediately after thawing.

### REPLICATION INCOMPETENT RECOMBINANT RETROVIRAL PARTICLES

In any of the embodiments disclosed herein, the methods can include a step of transducing the activated T cells and/or NK cells with replication incompetent recombinant retroviral particles including one or more nucleic acids to generate transduced T cells and/or NK cells. In some embodiments, the one or more nucleic acids can encode one or more proteins that are then expressed in the transduced T cells and/or NK cells, for example, a chimeric antigen receptor (CAR). The replication incompetent recombinant retroviral particles used to transduce the T cells and/or NK cells in the methods provided herein can be made according to methods known in the art. As disclosed herein, replication incompetent recombinant retroviral particles are a common tool for gene delivery (Miller, Nature (1992) 357:455-460). The ability of replication incompetent recombinant retroviral particles to deliver an unrearranged nucleic acid sequence into a broad range of rodent, primate, and human somatic cells makes replication incompetent recombinant retroviral particles well suited for transferring genes to a cell. In some embodiments, the replication incompetent recombinant retroviral particles can be derived from the Alpharetrovirus genus, the Betaretrovirus genus, the Gammaretrovirus genus, the Deltaretrovirus genus, the Epsilonretrovirus genus, the Lentivirus genus, or the Spumavirus genus. There are many retroviruses suitable for use in the methods disclosed herein. For example, murine leukemia virus (MLV), human immunodeficiency virus (HIV), equine infectious anaemia virus (EIAV), mouse mammary tumor virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) can be used. A detailed list of retroviruses may be found in Coffin et al (“Retroviruses” 1997 Cold Spring Harbor Laboratory Press Eds: J M Coffin, S M Hughes, H E Varmus pp 758-763). Details on the genomic structure of some retroviruses may be found in the art. By way of example, details on HIV may be found from the NCBI Genbank (i.e. Genome Accession No. AF033819).

In illustrative embodiments, the replication incompetent recombinant retroviral particles can be derived from a recombinant retrovirus from the Lentivirus genus and can be replication incompetent recombinant lentiviral particles. In some embodiments, the recombinant retrovirus can be derived from HIV, SIV, or FIV. In further illustrative embodiments, the recombinant retrovirus can be derived from the human immunodeficiency virus (HIV) in the Lentivirus genus. Lentiviruses are complex retroviruses which, in addition to the common retroviral genes gag, pol and env, contain other genes with regulatory or structural function. The higher complexity enables the lentivirus to modulate the life cycle thereof, as in the course of latent infection. A typical lentivirus is the human immunodeficiency virus (HIV), the etiologic agent of AIDS. In vivo, HIV can infect terminally differentiated cells that rarely divide, such as lymphocytes and macrophages.

In some embodiments, the replication incompetent recombinant retroviral particles can be grown in a culture in a medium which is specific for replication incompetent recombinant retroviral particle manufacturing. Any suitable growth media and/or supplements for growing replication incompetent recombinant retroviral particles can be used in the replication incompetent recombinant retroviral particle inoculum in accordance with the methods described herein. According to some aspects, the replication incompetent recombinant retroviral particles can then be added to the media during the transduction step.

The replication incompetent recombinant retroviral particles can be produced using mammalian cell lines according to methods known in the art. Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL1O), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, Hut-78, Jurkat, HL-60, NK cell lines (e.g., NKL, NK92, and YTS), and the like. In some instances, the cell is not an immortalized cell line, but is instead a cell (e.g., a primary cell) obtained from an individual or an ex vivo cell. For example, in some embodiments, the cell is an immune cell obtained from an individual. As another example, the cell is a stem cell or progenitor cell obtained from an individual.

### NUCLEIC ACIDS ENCODING CHIMERIC ANTIGEN RECEPTORS

In illustrative embodiments, the present disclosure provides methods for transducing T cells and/or NK cells with one or more nucleic acids that include a nucleotide sequence. In some embodiments, the one or more nucleic acids can include a nucleotide sequence that encodes a CAR. A nucleic acid including a nucleotide sequence encoding the CAR will in some embodiments be DNA, including, e.g., a recombinant expression vector. A nucleic acid including a nucleotide sequence encoding a CAR will in some embodiments be RNA, e.g., in vitro synthesized RNA.

In some embodiments, a nucleic acid provides for production of a CAR, e.g., in a mammalian cell. In other embodiments, a subject nucleic acid provides for amplification of the CAR-encoding nucleic acid.

A nucleotide sequence encoding a CAR can be operably linked to a transcriptional control element, e.g., a promoter, and enhancer, etc. Suitable promoter and enhancer elements are known in the art. For expression in a bacterial cell, suitable promoters include, but are not limited to, lad, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Suitable reversible promoters, including reversible inducible promoters are known in the art. Such reversible promoters can be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some instances, the locus or construct or trans gene containing the suitable promoter is irreversibly switched through the induction of an inducible system. Suitable systems for induction of an irreversible switch are well known in the art, e.g., induction of an irreversible switch can make use of a Cre-lox-mediated recombination (see, e.g., Fuhrmann-Benzakein, et al., PNAS (2000) 28:e99. Any suitable combination of recombinase, endonuclease, ligase, recombination sites, etc. known to the art can be used in generating an irreversibly switchable promoter. Methods, mechanisms, and requirements for performing site-specific recombination, disclosed elsewhere herein, find use in generating irreversibly switched promoters and are well known in the art, see, e.g., Grindley et al. (2006) Annual Review of Biochemistry, 567-605 and Tropp (2012) Molecular Biology (Jones & Bartlett Publishers, Sudbury, Mass.).

In some embodiments, a CAR is expressed from a promoter active in a T cell and/or an NK cell. For methods and compositions provided herein, a skilled artisan will recognize that promoters are known that are active in T cells and/or NK cells and can be used to express a first engineered signaling polypeptide or a second engineered signaling polypeptide, or any component thereof. In illustrative embodiments, such a promoter is not active in a packaging cell line, such as a packaging cell line used to make a retrovirus such as a lentivirus, used for transduction in methods provided herein. In some embodiments, the promoter is the EFla promoter or the murine stem cell virus (MSCV) promoter (Jones et al., Human Gene Therapy (2009) 20: 630-40). In illustrative embodiments, the promoter is the T cell specific CD3 zeta promoter.

In illustrative embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90:7739; and Marodon et al. (2003) Blood 101:3416. As another example, a CD8 gene promoter can be used. NK cell-specific expression can be achieved by use of a Neri (p46) promoter; see, e.g., Eckelhart et al. (2011) Blood 117:1565.

A nucleotide sequence encoding a CAR can be present in an expression vector and/or a cloning vector. Where a CAR includes two separate polypeptides, nucleotide sequences encoding the two polypeptides can be cloned in the same or separate vectors. An expression vector can include a selectable marker, an origin of replication, and other features that provide for replication and/or maintenance of the vector. Suitable expression vectors include, e.g., plasmids, viral vectors, and the like.

Large numbers of suitable vectors and promoters are known to those of skill in the art; many are commercially available for generating a subject recombinant constructs. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host can be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno- associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90: 10613-10617); SV40; herpes simplex virus; gamma retrovirus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

As noted above, in some embodiments, a nucleic acid including a nucleotide sequence encoding a CAR will in some embodiments be RNA, e.g., in vitro synthesized RNA. Methods for in vitro synthesis of RNA are known in the art; any known method can be used to synthesize RNA including a nucleotide sequence encoding a CAR. Methods for introducing RNA into a host cell are known in the art. See, e.g., Zhao et al. (2010) Cancer Res. 15:9053. Introducing RNA including a nucleotide sequence encoding a CAR into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding a CAR.

### CHIMERIC ANTIGEN RECEPTORS

In some embodiments, the replication incompetent recombinant retroviral particles used to transduce the T cells and/or NK cells can include one or more nucleic acids that include one or more transcriptional units that encode one or more chimeric antigen receptors (CARs). Throughout the present disclosure, a CAR or a polynucleotide encoding a CAR, which, for simplicity, is referred to herein as “CAR”. In some embodiments, a CAR includes any combination of the following: an extracellular antigen-specific targeting region (ASTR), a stalk, a transmembrane domain, an intracellular activating domain, and a modulatory domain (such as a co-stimulatory domain). In some embodiments, a CAR can include: a) at least one antigen-specific targeting region (ASTR); b) a transmembrane domain; and c) an intracellular activating domain.

### Antigen-Specific Targeting Region (ASTR)

In some embodiments, a CAR can include a member of a specific binding pair, which is typically an ASTR, sometimes called an antigen binding domain herein. Specific binding pairs include, but are not limited to, antigen-antibody binding pairs; ligand-receptor binding pairs; and the like. Thus, a member of a specific binding pair suitable for use in a CAR can include an ASTR that is an antibody, an antigen, a ligand, a receptor binding domain of a ligand, a receptor, a ligand binding domain of a receptor, and an affibody. An ASTR suitable for use in a CAR can be any antigen-binding polypeptide. In certain embodiments, the ASTR can be an antibody such as a full-length antibody, a single-chain antibody, an Fab fragment, an Fab′ fragment, an (Fab′)2 fragment, an Fv fragment, and a divalent single-chain antibody or a diabody. In some embodiments, the ASTR is a single chain Fv (scFv). In some embodiments, the heavy chain is positioned N-terminal of the light chain in the ASTR. In other embodiments, the light chain is positioned N-terminal of the heavy chain in the ASTR. In any of the embodiments disclosed herein, the heavy and light chains can be separated by a linker as discussed in more detail herein. In any of the disclosed embodiments, the heavy or light chain can be at the N-terminus of the ASTR and is typically C-terminal of another domain, such as a signal sequence or peptide.

Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use with the CAR and methods using the CAR. In some instances, T cell receptor (TCR) based recognition domains such as single chain TCR (scTv, single chain two-domain TCR containing VαVβ) are also suitable for use.

An ASTR suitable for use in a CAR can have a variety of antigen-binding specificities. In some embodiments, the antigen-binding domain is specific for an epitope present in an antigen that is expressed by (synthesized by) a target cell. In one example, the target cell is a cancer cell associated antigen. The cancer cell associated antigen can be an antigen associated with, e.g., a breast cancer cell, a B cell lymphoma, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer cell associated antigen may also be expressed by a non-cancerous cell.

Non-limiting examples of antigens to which an ASTR can bind include, e.g., CD19, CD20, CD38, CD30, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, Ax1, Ror2, and the like.

In some embodiments, a member of a specific binding pair suitable for use in a CAR can be an ASTR that is a ligand for a receptor. Ligands include, but are not limited to, cytokines (e.g., IL-13, etc.); growth factors (e.g., heregulin; vascular endothelial growth factor (VEGF); and the like); an integrin-binding peptide (e.g., a peptide comprising the sequence Arg-Gly-Asp); and the like.

Where the member of a specific binding pair in a CAR is a ligand, the CAR can be activated in the presence of a second member of the specific binding pair, where the second member of the specific binding pair is a receptor for the ligand. For example, where the ligand is VEGF, the second member of the specific binding pair can be a VEGF receptor, including a soluble VEGF receptor.

As noted above, in some embodiments, the member of a specific binding pair that is included in a CAR is an ASTR that is a receptor, e.g., a receptor for a ligand, a co-receptor, etc. The receptor can be a ligand-binding fragment of a receptor. Suitable receptors include, but are not limited to, a growth factor receptor (e.g., a VEGF receptor); a killer cell lectin-like receptor subfamily K, member 1 (NKG2D) polypeptide (receptor for MICA, MICB, and ULB6); a cytokine receptor (e.g., an IL-13 receptor; an IL-2 receptor; etc.); CD27; a natural cytotoxicity receptor (NCR) (e.g., NKP30 (NCR3/CD337) polypeptide (receptor for HLA-B-associated transcript 3 (BAT3) and B7-H6); etc.); etc.

### Microenvironment Restricted Biologic CARs (MRB-CARs)

In some embodiments, CARs made by methods of the present disclosure are microenvironment restricted. This property is typically the result of the microenvironment restricted nature of the ASTR domain of the CAR. Thus, CARs of the present disclosure can have a lower binding affinity or, in illustrative embodiments, can have a higher binding affinity to one or more target antigens under a condition(s) in a target microenvironment than under a condition in a normal physiological environment. These CARs can be referred to as microenvironment restricted biologic CARs, or MRB-CARs, or, in some instances, as conditionally active biologic CARs, or CAB-CARs. In exemplary embodiments, MRB-CARs made using methods provide herein, have a lower binding affinity, or in certain illustrative embodiments, a higher binding affinity in a tumor microenvironment.

Methods are available for identifying microenvironment restricted antibodies that can be used to make microenvironment restricted antibody fragments for ASTRs. For example, microenvironment restricted ASTRs can be identified from polypeptide library screens with or without mutating/evolving members of the library before screening or panning and with or without mutating/evolving during or between optional repeated rounds of screening or panning Exemplary methods for identifying microenvironment restricted antibodies, antibody fragments, and ASTRs are provided in WO2017/165245. In some embodiments, MRB-CARs can be obtained by identifying a VH and/or VL of an antibody that was identified under physiologic conditions (i.e. parent, “wild type” or “wt” antibody). Antibodies can then be mutated and tested (evolved). A skilled artisan can utilize the method for identifying conditionally active antibodies disclosed in U.S. Pat. No. 8,709,755 to identify additional conditionally active antibodies and antibody fragments that can be used in ASTRs for MRB-CARs of the present disclosure. To alter the binding specificity of a starting point (“wt” antibody), it is reasonable to expect that mutating either or both the VH and VL could lead to microenvironment restricted activity in a MRB-CAR. However, to generate a microenvironment restricted antibody, both the VH or the VL are typically identified under physiologic conditions, and then either the VH or the VL, but typically not both, is mutated and tested in non-physiologic conditions, such as a tumor microenvironment, to generate a conditionally active antibody. In certain non-limiting illustrative embodiments, an MRB-CAR used in any of the embodiments provided herein, is an anti-Ax1 MRB-CAR or anti-Ror2 MRB-CAR.

Normal physiological conditions can include those of temperature, pH, osmotic pressure, osmolality, oxidative stress, and electrolyte concentration that would be considered within a normal range at the site of administration, or at the tissue or organ at the site of action, to a subject. An aberrant condition is that which deviates from the normally acceptable range for that condition. In one aspect, a microenvironment restricted ASTR (i.e. polypeptide) is virtually inactive at normal conditions but is active at other than normal conditions at a level that is equal or greater than at normal conditions. For example, in one aspect, the microenvironment restricted ASTR is virtually inactive at body temperature, but is active at lower temperatures. In another aspect, the microenvironment restricted ASTR is reversibly or irreversibly inactivated at the normal conditions.

### Stalk and Hinge Regions

In some embodiments, the CAR can include a stalk which is located in the portion of the CAR lying outside the cell and interposed between the ASTR and the transmembrane domain Stalks can include immunoglobulin hinge region amino acid sequences that are known in the art; see, e.g., Tan et al. (1990) Proc. Natl. Acad. Sci. USA 87:162; and Huck et al. (1986) Nucl. Acids Res. 14:1779. In a CAR, the stalk employed allows the ASTR, and typically the entire CAR, to retain increased binding to a target antigen. In some embodiments, the stalk of a CAR can include at least one cysteine. The stalk region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

The stalk can include a hinge region with an amino acid sequence of a human IgG1, IgG2, IgG3, or IgG4, hinge region. The stalk can include one or more amino acid substitutions and/or insertions and/or deletions compared to a wild-type (naturally-occurring) hinge region. For example, His229 of human IgG 1 hinge can be substituted with Tyr, so that the stalk includes the sequence EPKSCDKTYTCPPCP (see, e.g., Yan et al. (2012) J. Biol. Chem. 287:5891). The stalk can include an amino acid sequence derived from human CD8.

### Transmembrane Domain

A CAR can include transmembrane domains for insertion into a eukaryotic cell membrane. The transmembrane domain can be interposed between the ASTR and the co-stimulatory domain The transmembrane domain can be interposed between the stalk and the intracellular activating domain (IAD) or co-stimulatory domain (CSD), such that the chimeric antigen receptor can include, in order from the amino terminus (N-terminus) to the carboxyl terminus (C-terminus): an ASTR; a stalk; a transmembrane domain; and an activating domain. Any transmembrane (TM) domain that provides for insertion of a polypeptide into the cell membrane of a eukaryotic (e.g., mammalian) cell is suitable for use in aspects and embodiments disclosed herein. As non-limiting examples, a transmembrane domain can have at least 80, 90, or 95% sequence identity to the CD8 beta transmembrane domain, the CD4 transmembrane domain, the CD3 zeta transmembrane domain, the CD28 transmembrane domain, the CD134 transmembrane domain, or the CD7 transmembrane domain.

### Intracellular Activating Domain

Intracellular activating domains (IADs) suitable for use in a CAR typically induce, upon activation, the production of one or more cytokines; increased cell death; and/or increased proliferation of CD8⁺ T cells, CD4⁺ T cells, natural killer T cells, γδT cells, and/or neutrophils. Intracellular activating domains can also be referred to as activating domains or activation domains herein. In some embodiments, the IAD can include at least one (e.g., one, two, three, four, five, six, etc.) ITAM motifs as described below. In some embodiments, the IAD can include DAP10/CD28 type signaling chains. As non-limiting examples, an IAD of a CAR can be a CD3Z, CD3D, CD3E, CD3G, CD79A, DAP12, FCERIG, DAP10/CD28, or ZAP70 activating domain

IADs suitable for use in a CAR can include immunoreceptor tyrosine-based activation motif (ITAM)-containing intracellular signaling polypeptides. An ITAM motif is YX₁X₂L/I, where X₁ and X₂ are independently any amino acid. In some embodiments, the intracellular activating domain of a CAR includes 1, 2, 3, 4, or 5 ITAM motifs. In some embodiments, an ITAM motif is repeated twice in an intracellular activating domain, where the first and second instances of the ITAM motif are separated from one another by 6 to 8 amino acids, e.g., (YX₁X₂L/I)(X₃)ₙ(YX₁X₂L/I), where n is an integer from 6 to 8, and each of the 6-8 X₃ can be any amino acid. In some embodiments, the IAD of a CAR includes 3 ITAM motifs.

A suitable IAD can be an ITAM motif-containing portion that is derived from a polypeptide that contains an ITAM motif. For example, a suitable IAD can be an ITAM motif-containing domain from any ITAM motif-containing protein. Thus, a suitable IAD need not contain the entire sequence of the entire protein from which it is derived. Examples of suitable IADs and ITAM motif-containing polypeptides include, but are not limited to: T cell surface glycoprotein CD3Z (also known as CD3 zeta chain, T cell receptor T3 zeta chain, CD247, CD3-ZETA, CD3H, CD3Q, T3Z, TCRZ, etc.); CD3D (also known as CD3 delta, CD3-DELTA, T3D, CD3 antigen delta subunit, CD3d antigen delta polypeptide (TiT3 complex) OKT3 delta chain, T cell receptor T3 delta chain, T cell surface glycoprotein CD3 delta chain, etc.); CD3E (also known as CD3 epsilon chain, T cell surface antigen T3/Leu-4 epsilon chain, T cell surface glycoprotein CD3 epsilon chain, AI504783, CD3, CD3epsilon, T3e, etc.); CD3G (also known T cell surface glycoprotein CD3 gamma chain, T cell receptor T3 gamma chain, CD3-GAMMA, T3G, gamma polypeptide (TiT3 complex), etc.); CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; antigen receptor complex-associated protein alpha chain, etc.); DAP12 (also [symbol] known as TYROBP; TYRO protein tyrosine kinase binding protein; KARAP; PLOSL; DNAX-activation protein 12; KAR-associated protein; TYRO protein tyrosine kinase [symbol] binding protein; killer activating receptor associated protein; killer-activating receptor- [symbol] associated protein; etc.); and FCERIG (also known as FCRG; Fc epsilon receptor I gamma chain; Fc receptor gamma-chain; fc-epsilon RI-gamma; fcRgamma; fceRl gamma; high affinity immunoglobulin epsilon receptor subunit gamma; immunoglobulin E receptor, high affinity, gamma chain; etc.).

### Modulatory Domains

Modulatory domains can change the effect of the IAD in a CAR, including enhancing or dampening the downstream effects of the IAD or changing the nature of the response. Modulatory domains suitable for use in a CAR of the present disclosure include co-stimulatory domains (CSDs). A modulatory or co-stimulatory domain suitable for inclusion in the CAR can have a length of from about 30 amino acids to about 70 amino acids (aa), e.g., a modulatory domain can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, modulatory domain can have a length of from about 70 aa to about 100 aa, from about 100 aa to about 200 aa, or greater than 200 aa.

CSDs typically enhance and/or change the nature of the response to activation of an activation domain CSDs suitable for use in a CAR are generally polypeptides derived from receptors. In some embodiments CSDs homodimerize. In some embodiments a CSD can be an intracellular portion of a transmembrane protein (i.e., the CSD can be derived from a transmembrane protein). Non-limiting examples of suitable co-stimulatory polypeptides include, but are not limited to, 4-1BB (also known as TNFRSF9; CD137; CDw137; ILA; etc.), CD27 (also known as S 152, T 14, TNFRSF7, and Tp55), CD28 (also known as Tp44), CD28 deleted for Lck binding (ICA), ICOS (also known as AILIM, CD278, and CVID1), OX40 (also known as TNFRSF4, RP5-902P8.3, ACT35, CD134, OX-40, TXGP1L), BTLA (also known as BTLA1 and CD272), CD30 (also known as TNFRSF8, D1S166E, and Ki-1), GITR (also known as TNFRSF18, RP5-902P8.2, AITR, CD357, and GITR-D), and HVEM (also known as TNFRSF14, RP3-395M20.6, ATAR, CD270, HVEA, HVEM, LIGHTR, and TR2). For example, a CSD can have at least 80%, 90%, or 95% sequence identity to the CSD of 4-1BB (CD137), CD27, CD28, CD28 deleted for Lck binding (ICΔ), ICOS, OX40, BTLA, CD27, CD30, GITR, or HVEM.

### Linker

In some embodiments, the CAR can include a linker between any two adjacent domains. For example, a linker can be between the transmembrane domain and a CSD. As another example, the ASTR can be an antibody and a linker can be between the heavy chain and the light chain. As another example, a linker can be between the ASTR and the transmembrane domain and a co-stimulatory domain. As another example, a linker can be between the co-stimulatory domain and the intracellular activating domain of the second polypeptide. As another example, the linker can be between the ASTR and the intracellular signaling domain.

The linker peptide may have any of a variety of amino acid sequences. Proteins can be joined by a spacer peptide, generally of a flexible nature, although other chemical linkages are not excluded. A linker can be a peptide of between about 1 and about 100 amino acids in length, or between about 1 and about 25 amino acids in length. These linkers can be produced by using synthetic, linker-encoding oligonucleotides to couple the proteins. Peptide linkers with a degree of flexibility can be used. The linking peptides may have virtually any amino acid sequence, bearing in mind that suitable linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids. Exemplary flexible linkers include glycine polymers (G)ₙ, glycine-serine polymers (including, for example, (GS)ₙ, GSGGSₙ, GGGSₙ, and GGGGSₙ where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). The ordinarily skilled artisan will recognize that design of a peptide conjugated to any elements described above can include linkers that are all or partially flexible, such that the linker can include a flexible linker as well as one or more portions that confer less flexible structure.

### Recognition or Elimination Domains

Any of the replication incompetent recombinant retroviral particles provided herein can include nucleic acids that encode a recognition or elimination domain as part of, or separate from, nucleic acids encoding any of the engineered signaling polypeptides provided herein. Thus, any of the engineered signaling polypeptides provided herein, can include a recognition or elimination domain For example, any of the CARs disclosed herein can include a recognition or elimination domain Moreover, a recognition or elimination domain can be expressed together with, or even fused with any of the lymphoproliferative elements disclosed herein. The recognition or elimination domains are expressed on the T cell and/or NK cell but are not expressed on the replication incompetent recombinant retroviral particles.

In some embodiments, the recognition or elimination domain can be derived from herpes simplex virus-derived enzyme thymidine kinase (HSV-tk) or inducible caspase-9. In some embodiments, the recognition or elimination domain can include a modified endogenous cell-surface molecule, for example as disclosed in U.S. Pat. No. 8,802,374. The modified endogenous cell-surface molecule can be any cell-surface related receptor, ligand, glycoprotein, cell adhesion molecule, antigen, integrin, or cluster of differentiation (CD) that is modified. In some embodiments, the modified endogenous cell-surface molecule is a truncated tyrosine kinase receptor. In one aspect, the truncated tyrosine kinase receptor is a member of the epidermal growth factor receptor (EGFR) family (e.g., ErbB1, ErbB2, ErbB3, and ErbB4. In some embodiments, the recognition domain can be a polypeptide that is recognized by an antibody that recognizes the extracellular domain of an EGFR member. In some embodiments, the recognition domain can be at least 20 contiguous amino acids of an EGFR family member, or for example, between 20 and 50 contiguous amino acids of an EGFR family member. For example, SEQ ID NO:78, is an exemplary polypeptide that is recognized by, and under the appropriate conditions bound by an antibody that recognizes the extracellular domain of an EGFR member. Such extracellular EGFR epitopes are sometimes referred to herein as eTags. In illustrative embodiments, such epitopes are recognized by commercially available anti-EGFR monoclonal antibodies.

### METHODS USING CELLS PRODUCED BY METHODS PROVIDED HEREIN

In some embodiments, the present disclosure provides methods for genetically modifying and expanding T cells and/or NK cells that can be used in various treatment methods. In some embodiments, the T cells and/or NK cells can be genetically modified to express a chimeric antigen receptor (CAR) and as such, can be used in CAR-T therapy. CARs and MRB-CARs are not limited to use in treatment of tumors, but rather could be appropriate for use in one or more indication including the treatment of circulatory disorders, arthritis, multiple sclerosis, autoimmune disorders, cancer, dermatologic disorders and use in various diagnostic formats.

In any of the embodiments disclosed herein, before introducing the genetically modified T cells and/or NK cells into a subject, the subject can be lymphodepleted according to methods known in the art. Typically, a lymphodepleting agent is administered to the subject to lymphodeplete the subject. In illustrative embodiments herein, the subject is lymphodepleted at a lymphodepletion timepoint reached when cell expansion reaches an expansion progress criteria. The expansion progress criteria can be selected from a lactate concentration in the cell expansion media exceeding a threshold value, for example 1 mM, 2 mM, 2.5 mM, 5 mM, or 10 mM. In other embodiments, the expansion progress criteria is a cell expansion level that exceeds a certain threshold level of (fold) expansion, such as 2, 3, 4, 5, 10, 15, or 20-fold expansion. In other embodiments, the expansion progress criteria is a cell density during the expanding exceeding a threshold value. In yet additional embodiments, the expansion progress criteria is a predetermined number of days of expansion. Such illustrative embodiments have the advantage over prior methods of assuring that T cell expansion and lymphodepletion of the subject to which the expanded T cells will be administered, are coordinated and synchronized.

Using the methods provided herein, T cells and/or NK cells can be transduced with one or more nucleic acids that include a nucleotide sequence encoding a CAR. A CAR, when present on a T cell and/or an NK cell, can mediate cytotoxicity toward a target cell. A CAR can bind to an antigen present on a target cell, thereby mediating killing of a target cell by the T cell and/or an NK cell genetically modified to produce the CAR. The ASTR of the CAR binds to an antigen present on the surface of a target cells. Such methods include CAR-T therapy.

Target cells include, but are not limited to, cancer cells. Thus, the present disclosure provides methods of killing, or inhibiting the growth of, a target cancer cell, the method involving contacting a cytotoxic immune effector cell (e.g., a cytotoxic T cell, or an NK cell) that is genetically modified to produce a CAR, such that the T cell and/or NK cell recognizes an antigen present on the surface of a target cancer cell, and mediates killing of the target cell.

The present disclosure provides a method of treating cancer in a subject having a cancer. As such the present disclosure provides methods for adoptive cellular therapy against cancer. Accordingly, in one aspect the method includes the following: a. introducing an expression vector configured to express a polynucleotide sequence encoding a CAR as provided herein, into PBMCs obtained from the subject to produce a genetically engineered cytotoxic cell (such as a T cell and/or NK cell); and b. administering the genetically engineered cytotoxic cell to the subject. The CAR can be any of the CARs disclosed herein. The expression vector encoding a CAR can be introduced into PBMCs by transducing T cells and/or NK cells with the vector according to the methods provided herein. In certain illustrative embodiments, the vector can be a replication incompetent recombinant retroviral particle that in some embodiments can be a replication incompetent recombinant lentiviral particle. In some embodiments, the T cells and/or NK cells of the subject are transduced with a CAR disclosed herein and the transduced T cells and/or NK cells are then administered to the subject.

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be amenable to therapy by a method disclosed herein include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be amenable to therapy by a method disclosed herein include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; non-Hodgkin's lymphoma, and the like.

Other cancers that can be amenable to treatment according to the methods disclosed herein include atypical meningioma (brain), islet cell carcinoma (pancreas), medullary carcinoma (thyroid), mesenchymoma (intestine), hepatocellular carcinoma (liver), hepatoblastoma (liver), clear cell carcinoma (kidney), and neurofibroma mediastinum.

In some embodiments, a T cell and/or NK cell expressing a CAR cell is administered as an adjuvant therapy to a standard cancer therapy. Standard cancer therapies include surgery (e.g., surgical removal of cancerous tissue), radiation therapy, bone marrow transplantation, chemotherapeutic treatment, antibody treatment, biological response modifier treatment, and certain combinations of the foregoing. Standard cancer therapies are well-known in the art. Radiation therapy includes, but is not limited to, x-rays or gamma rays that are delivered from either an externally applied source such as a beam, or by implantation of small radioactive sources.

### GENETICALLY ENGINEERED T CELLS AND/OR NK CELLS AND CELL POPULATIONS

In some aspects, provided herein is a genetically engineered and/or isolated T cell or NK cell, or a population of genetically engineered T cells and/or NK cells, or a population of genetically engineered NK cells, or in illustrative embodiments, a population of genetically engineered T cells produced using the methods provided herein. Such a cell or population can be in a chemically defined media, such as a media used in a method provided herein. Such a cell or population is typically found in a media comprising IL-2 and in some embodiments IL-7, typically IL-2 and IL-7 that is not from the same subject as the original source of the T cells and/or the NK cells, and in further illustrative embodiments, recombinant IL-2 and IL-7. In some embodiments, the population of cells produced by a method herein, is over 75%, 80%, 85%, 90%, or 95% T cells, including NK T cells. In some embodiments, the population of cells produced by a method herein includes 10% or less, 5% or less, 4% or less, 3% or less, 2% or less, 1% or less NK cells, or between 0.2 , 03, 0.4, or 0.5% NK cells on the low end of the range, and 0.6, 0.7. 0.8, 0.9, 1, 1.5, 2, 2.5, 5, or 10% on the high end of the range.

In some embodiments, the population of genetically engineered T cells provided by a method herein is between 5% and 20% NK T cells. In some embodiments, the population of genetically engineered T cells provided by a method herein is between 5% and 20% NK T cells, between 5% to 30% CD4+ T cells and 60 to 90% CD8+ T cells. In certain embodiments, the population of T cells has a ratio of 1:1 or in illustrative embodiments, at least twice, 2.5 times, or three times as many CD8 positive cells as CD4 positive cells. The ratio of CD8 to CD4 cells in the population of genetically engineered T cells can be shifted towards 1:1 by using both anti-CD3 and anti-CD28 in the activation reaction mixture, for example.

In some embodiments, the population of genetically engineered T cells comprises over 75, 80, 85, 90, or 95% T cells, and between 30 and 90%, or between 30 and 80%, or between 30 and 75% or between 30 and 70% genetically engineered T cells. In some embodiments, the population of genetically engineered T cells comprises over 70, 75, 80, or 85 T cells on the low side of the range, and between 90, 95, 96, 97, 98, 99, 99.5, 99.9, or 100% T cells (genetically engineered and not genetically engineered) on the high side of the range. In some embodiments, the population of genetically engineered T cells comprises between 20, 25, 30, 35, or 40% on the low side of the range, and 45, 50, 60, 70, and 75% genetically engineered T cells.

Such genetically engineered T cells and/or NK cells in illustrative embodiments, are engineered such that their genomes comprise nucleic acids encoding a CAR. Thus, the genetically engineered T cells and/or NK cells in some embodiments express a CAR. In some embodiments the genetically engineered cells are T cells. In some embodiments, the population of genetically engineered T cells comprises between 20, 25, 30, 35, or 40% on the low side of the range, and 45, 50, 60, 70, and 75% on the high side of the range, genetically engineered T cells that express a CAR. The CAR can comprise any of the CAR components disclosed herein. In some embodiments, the T cells and/or NK cells express an elimination domain In some embodiments, the elimination domain is an e-TAG.

The genetically engineered T cells and/or NK cells, or a population thereof, provided herein in some embodiments are in a chemically defined culture media, which can be, for example within a chamber of a closed cell processing system, or within a cell collection bag. In other embodiments, the genetically engineered T cells and/or NK cells, or a population thereof, are within a commercial cryopreservative media, or a media comprised in part of such a commercial cryopreservative media.

### EXEMPLARY EMBODIMENTS

Some of the embodiments provided herein include the following:

Embodiment 1A1. A method for transducing T cells and/or NK cells from isolated blood, comprising:

a) enriching peripheral blood mononuclear cells (PBMCs) to isolate PBMCSs comprising T cells and/or NK cells from isolated blood;

b) activating T cells and/or NK cells of the isolated PBMCs under effective conditions within a closed system and without enriching T cells and/or NK cells from other PBMCs, comprising an effective amount of anti-CD3 antibody in solution; and

c) transducing the activated T cells and/or NK cells with replication incompetent recombinant retroviral particles under effective conditions, thereby producing genetically modified T cells and/or NK cells, wherein the activating and transducing are performed within the same closed system without washing the cells between the activating and transducing.

Embodiment 1A2. The method of Embodiment 1A1, further comprising expanding the genetically modified T cells and/or NK cells in cell expansion media to a volume exceeding 150 ml and an expansion completion selection criteria selected from a lactate concentration exceeding 10 mM, at least a 5-fold expansion of T cells and/or NK cells, and at least 10 days in cell expansion media.

Embodiment 1A3. The method of Embodiment 1A1, wherein the expanding is performed within the same chamber of the same closed system as the activating and transducing.

Embodiment 1A4. The method of Embodiment 1A2, wherein the expanding is performed without washing the cells between the transducing and the expanding.

Embodiment 1A5. The method of Embodiment 1A2, wherein the expansion completion selection criteria is at least a 10-fold expansion of T cells and/or NK cells.

Embodiment 1B1. A method for transducing T cells and/or NK cells from isolated blood, comprising:

a) enriching peripheral blood mononuclear cells (PBMCs) to isolate PBMCs comprising T cells and/or NK cells from isolated blood;

b) activating T cells and/or NK cells of the isolated PBMCs under effective conditions within a chamber of a closed system, comprising an effective amount of anti-CD3 antibody and/or an effective amount of anti-CD28;

c) transducing the activated T cells and/or NK cells with replication incompetent recombinant retroviral particles under effective conditions, thereby producing genetically modified T cells and/or NK cells; and

d) expanding the genetically modified T cells and/or NK cells in cell expansion media to a volume exceeding 150 ml and an expansion completion selection criteria selected from a lactate concentration exceeding 10 mM, at least a 10-fold expansion of T cells and/or NK cells, and at least 4 days in cell expansion media, wherein the activating, transducing, and expanding are performed within the chamber without washing the cells between or during the activating, transducing, and expanding.

Embodiment 1C1. A method for transducing T cells and/or NK cells from isolated blood, comprising:

a) enriching peripheral blood mononuclear cells (PBMCs) to isolate PBMCs comprising T cells and/or NK cells from isolated blood;

b) activating T cells and/or NK cells of the isolated PBMCs under effective conditions within a closed system comprising an effective amount of anti-CD3 antibody and/or an effective amount of anti-CD28;

c) transducing the activated T cells and/or NK cells with replication incompetent recombinant retroviral particles under effective conditions, thereby producing genetically modified T cells and/or NK cells; and

d) expanding the genetically modified T cells and/or NK cells in cell expansion media to a volume exceeding 150 ml and an expansion completion selection criteria selected from a lactate concentration exceeding 10 mM, at least a 10-fold expansion of T cells and/or NK cells, and at least 10 days in cell expansion media, wherein anti-CD3 antibody and/or anti-CD28 is present in the cell expansion media comprising the expanded T cells and/or NK cells.

Embodiment 1D1. A method for transducing T cells and/or NK cells from isolated blood, comprising:

a) enriching peripheral blood mononuclear cells (PBMCs) to isolate PBMCs comprising T cells and/or NK cells from isolated blood;

b) activating T cells and/or NK cells of the isolated PBMCs under effective conditions within a closed system comprising a reaction mixture comprising base cell culture media and an effective amount of anti-CD3 antibody and/or an effective amount of anti-CD28;

c) transducing the activated T cells and/or NK cells with replication incompetent recombinant retroviral particles under effective conditions, thereby producing genetically modified T cells and/or NK cells, wherein the activating and transducing are performed within the same closed system and wherein the transducing is performed after the activating by adding the replication incompetent recombinant retroviral particles to the reaction mixture; and

d) expanding the genetically modified T cells and/or NK cells in cell expansion media to a volume exceeding 150 ml and an expansion completion selection criteria selected from a lactate concentration exceeding 10 mM, at least a 10-fold expansion of T cells and/or NK cells, and at least 10 days in cell expansion media, wherein the cell expansion media comprises the base cell culture media and supplemental N-acetyl cysteine (NAC) in addition to any NAC present in the base cell culture media, and wherein supplemental NAC is absent during the transducing.Embodiment 1D2. A method according to Embodiment 1C1 or 1D1, or according to any other Embodiment provided herein unless explicitly recited otherwise, wherein more than 1/1,000^{th}, 1/500^{th}, 1/250^{th}, 1/100^{th}, 1/50^{th}, or 1/20^{th} the concentration of anti-CD3 antibody and/or anti-CD28 antibody are present in the cell expansion media as are present in an activation reaction mixture in which the activating is performed.

Embodiment 2. The method of any one of Embodiments 1A2, 1B1, 1C1, or 1D1, or of any other Embodiment provided herein unless explicitly recited otherwise, wherein the expanding is performed without removing more than 1%, 2%, 5%, 10%, 15% or 20% of the cell expansion media at any time during the expanding.

Embodiment 3. The method of any one of Embodiments 1A2, 1C1, or 1D1, or of any other Embodiment provided herein, wherein the activating, transducing, and expanding are performed in the same chamber without removing the T cells and/or NK cells from the chamber between the start of the activating and the completion of at least 7 days of expanding the T cells and/or NK cells in cell expansion media.

Embodiment 4. A method according to any one of Embodiments 1A2, 1B1, or 1C1, or according to any other Embodiment provided herein unless recited otherwise, wherein N-acetyl cysteine (NAC) is added to the cell expansion media, wherein the cell expansion media comprises a concentration of NAC that is greater than the concentration of NAC present in a transduction reaction mixture in which the transduction reaction is performed.

Embodiment 5. A method according to Embodiment 4, wherein NAC is present in the cell expansion media at a concentration between 5 mM and 20 mM, 5 mM and 15 mM, 7.5 mM and 12.5 mM, or 9 mM and 11 mM greater than the concentration of NAC present in the transduction reaction mixture.

Embodiment 6. The method of any one of Embodiments 1A2, 1B1, 1C1, or 1D1, or of any other Embodiment provided herein, wherein aminobisphosphonates are not present in the media during the activating.

Embodiment 7. The method of any one of Embodiments 1A2, 1B1, 1C1, or 1D1, or of any other Embodiment provided herein, wherein serum is not present in the cell expansion media.

Embodiment 8. The method of Embodiment 4 or Embodiment 1D1, or any other Embodiment provided herein, wherein the PBMCs are isolated from other than a healthy subject.

Embodiment 9. The method of Embodiment 4, Embodiment 8, or Embodiment 1D1, or of any other Embodiment provided herein wherein the subject is not explicitly recited as having a certain disease, wherein the PBMCs are isolated from a subject diagnosed with cancer.

Embodiment 10. The method of any one of Embodiments 1A1, 1B1, 1C1, or 1D1, or of any other Embodiment provided herein, wherein the effective conditions for the activating step comprise a concentration of isolated PBMCs between 5×10⁴ PBMCs/ml and 4×10⁶ PBMCs/ml.

Embodiment 11. The method of any one of Embodiments 1A1, 1B1, 1C1, or 1D1, or of any other Embodiment provided herein, wherein the effective conditions for the activating step comprise incubating the isolated PBMCs for between 4 hours and 48 hours or between 6 hour and 24 hours or between 6 hours and 12 hours.

Embodiment 12. The method of any one of Embodiments 1A1, 1B1, 1C1, or 1D1, or any other Embodiment provided herein unless explicitly recited otherwise, wherein the activated cells comprise T cells.

Embodiment 13. The method of any one of Embodiments 1A2, 1B1, 1C1, or 1D1, or any other Embodiment unless explicitly stated otherwise, wherein the activating and the expanding occur in the presence of an effective amount of IL-2.

Embodiment 14. The method of Embodiment 13, wherein the effective amount of IL-2 is between 25 IU/ml and 299 IU/ml.

Embodiment 15. The method of Embodiment 13, wherein IL-2 is present at a concentration of below 300 international units/ml for the activating and expanding.

Embodiment 16. The method of Embodiment 13, wherein IL-2 is present in the cell culture media at the start of the expanding and is added to the cell expansion media at least two times during the expanding.

Embodiment 17. The method of Embodiment 13, wherein IL-2 is present in the cell culture media at the start of the expanding and is added to the cell expansion media every two to three days during the expanding.

Embodiment 18. The method of Embodiment 13, wherein IL-2 is present in the cell culture media at the start of the expanding and is added to the cell expansion media between days two and five of the expanding.

Embodiment 19. The method of any one of Embodiments 15 to 18, wherein IL-7 is present in the cell culture media during the expanding step.

Embodiment 20. The method of Embodiment 14 or 15, wherein the T cells and/or the NK cells are expanded at least 20-fold from the number of T cells and/or NK cells in the activation step.

Embodiment 21. The method of Embodiment 14 or 15, wherein the T cells and/or the NK cells are expanded at least 25-fold from the number of T cells and/or NK cells in the activation step.

Embodiment 22. A method according to any one of the Embodiments provided herein, wherein the replication incompetent recombinant retroviral particles each comprise a retroviral genome comprising one or more nucleic acid sequences operatively linked to a promoter active in T cells and/or NK cells, wherein a first nucleic acid sequence of the one or more nucleic acid sequences encodes a chimeric antigen receptor (CAR) comprising:

a) an antigen-specific targeting region (ASTR),

b) a transmembrane domain, and

c) an intracellular activating domain.

Embodiment 23. The method of Embodiment 22, wherein the ASTR is a microenvironment restricted ASTR (MRB-ASTR). As will be understood, an MRB-ASTR exhibits an increased binding to one or more target antigens under a condition present in a target microenvironment than in the condition present in a normal physiological environment. In some embodiments, the MRB-ASTR binds to Ax1 or Ror2 under target conditions such as a pH of 6.7.

Embodiment 24. The method of Embodiment 23, wherein the condition is selected from the group consisting of: temperature, pH, osmotic pressure, osmolality, oxidative stress, and electrolyte concentration.

Embodiment 25. The method of Embodiment 24, wherein the condition is pH.

Embodiment 26. The method of any one of Embodiments 23 to 25, wherein the MRB-ASTR exhibits increased binding to its cognate target antigen at a pH of 6.7 versus a pH of 7.4.

Embodiment 27. A method according to any one of Embodiments 1B1, 1C1, or 1D1, or according to any other Embodiment provided herein unless explicitly recited otherwise, wherein the activating is performed in the presence of anti-CD3 antibody in solution.

Embodiment 28. The method of any one of Embodiments 1A1, 1B1, 1C1, or 1D1, or according to any other Embodiment provided herein unless explicitly recited otherwise, wherein the activating is performed in the absence of anti-CD3 antibody attached to a solid support and/or anti-CD28 attached to a synthetic solid support.

Embodiment 29. The method of any one of Embodiments 1A2, 1B1, 1C1, or 1D1, or according to any other Embodiment provided herein unless explicitly recited otherwise, wherein the effective conditions for the transducing comprise incubating the activated T cell and/or NK cell in the presence of the replication incompetent recombinant retroviral particles for between 6 hours and 36 hours before adding the cell expansion media.

Embodiment 30. The method of any one of Embodiments 1A2, 1B1, 1C1, or 1D1, or according to any other Embodiment provided herein unless explicitly recited otherwise, wherein the method further comprises harvesting the genetically modified T cells and/or NK cells after the expanding.

Embodiment 31. The method of Embodiment 30, wherein the harvesting is performed when a concentration of lactate in the cell expansion media reaches between 10 and 30 mM.

Embodiment 32. The method of Embodiment 30, wherein the harvesting is performed within 12 days of collecting the blood.

Embodiment 33. A method according to any one of Embodiments 1A2, 1B1, 1C1, or 1D1, or according to any other Embodiment provided herein unless explicitly recited otherwise, further comprising harvesting the expanded T cells and/or NK cells when the cells are expanded for between 10 and 14 days.

Embodiment 34. The method of Embodiment 30, wherein no more than 1%, 2%, 2.5%, 5%, or 10% of media is removed during performance of the method from the start of the activating until the beginning of the harvesting.

Embodiment 35. A method according to any one of Embodiments 1B1, 1C1, or 1D1, or according to any other Embodiment provided herein unless explicitly recited otherwise, wherein the method is performed without enriching T cells and/or NK cells from other PBMCs before the activating step.

Embodiment 36. The method of any one of Embodiments 1A2, 1B1, 1C1, or 1D1, or according to any other Embodiment provided herein unless explicitly recited otherwise, wherein the expanding is performed within a rigid cell culture container within the closed system that is permeable to gas.

Embodiment 37. The method of any one of Embodiments 1A1, 1B1, 1C1, or 1D1, or according to any other Embodiment provided herein unless explicitly recited otherwise, wherein recombinant human fibronectin is not present during the activating and/or transducing.

Embodiment 38. The method of Embodiment 36, wherein the activating, transducing, and expanding are performed within the same rigid cell culture container within the closed system.

Embodiment 39. The method of Embodiment 38, wherein the T cells and/or NK cells are not removed from the rigid cell culture container at any point from the beginning of the activating through completion of the expanding step.

Embodiment 40. The method of Embodiment 30, wherein the cells are harvested when the transduced T cells and/or NK cells are expanded at least 10 fold.

Embodiment 41. A method according to any one of the preceding Embodiments, or according to any other Embodiment provided herein unless explicitly recited otherwise, wherein the activated, transduced, and expanded cells comprise at least 60%, 70%, 75%, 80%, 85%, or 90% T cells.

Embodiment 42. A modified T cell produced by a method of any of the method Embodiments provided herein.

Embodiment 43. A modified NK cell produced by a method of any of the method Embodiments provided herein.

Embodiment 44. A method of any of Embodiments 30 to 33, further comprising cryopreserving the harvested genetically modified T cells and/or NK cells.

Embodiment 45. The method of Embodiment 44, wherein the cryopreserved genetically modified T cells and NK cells are thawed.

Embodiment 46. A method of any of Embodiments 30 to 33 and 45, further comprising introducing the harvested genetically modified T cells and/or NK cells into a subject.

Embodiment 47. The method of Embodiment 46, or according to any other Embodiment provided herein unless explicitly recited otherwise, further comprising collecting blood from the subject to obtain the isolated blood.

Embodiment 48. The method of Embodiment 47, wherein the harvested genetically modified T cells and/or NK cells are reintroduced into the subject from which the blood was collected.

Embodiment 49. The method of Embodiment 48, wherein the subject is lymphodepleted at a lymphodepletion timepoint reached when the expanding attains or exceeds an expansion progress criterion.

Embodiment 50. The method of Embodiment 49, wherein the expansion progress criterion is selected from a lactate concentration in the cell expansion media exceeding 1 mM, at least a 2-fold expansion of T cells and/or NK cells, or a predetermined number of days of expanding.

Embodiment 51. The method of Embodiment 49, wherein the subject is lymphodepleted when a lactate concentration in the cell expansion media exceeds 5 mM.

Embodiment 52. The method of Embodiment 49, wherein the subject is lymphodepleted when a lactate concentration of the cell expansion media exceeds 10 mM.

Embodiment 53. The method of Embodiment 49, wherein the subject is lymphodepleted when a lactate concentration of the cell expansion media exceeds 20 mM.

Embodiment 54. The method of Embodiment 49, wherein the subject is lymphodepleted when at least a 2-fold, 2.5-fold, 3-fold, 4-fold, or 5-fold expansion of cells, viable cells, PBMCs, T cells and NK, or T cells over the number of PBMCs present at the start of or during the activating or at the start of the transducing, is attained.

Embodiment 55. A method according to any of the method Embodiments provided herein unless otherwise recited, wherein the subject is afflicted with cancer.

Embodiment 56. The method of any of Embodiments 46 to 54, wherein the method is performed to treat a disease for which the subject is afflicted.

Embodiment 57. The method of Embodiment 56, wherein the disease is cancer.

Embodiment 58. A method according to any of the method Embodiments provided herein unless explicitly recited otherwise, wherein the transduction is performed without centrifugation.

Embodiment 59. A method according to any of the method Embodiments provided herein unless explicitly recited otherwise, wherein the activation is performed without centrifugation.

Embodiment 60. A method according to any of the method Embodiments provided herein unless explicitly recited otherwise, wherein the activation, transduction, and expansion are performed without centrifugation.

Embodiment 61. A method according to any of the method Embodiments provided herein unless explicitly recited otherwise, wherein the effective conditions for activating do not comprise anti-CD28.

Embodiment 62. A method according to any of the method Embodiments provided herein unless explicitly recited otherwise, wherein anti-CD28 in a soluble form or anti-CD28 attached to a synthetic solid support are not present during the activating step.

Embodiment 63. A method according to Embodiment 61, wherein between 45% and 95%, or between 50% and 95%, or between 55% and 90%, or between 60% and 90% of the expanded cells are CD8+ T cells.

Embodiment 64. A method according to Embodiment 61, wherein the expanded cells comprise at least 1.5 times, 2 times, 2.5 times, or 3 times as many CD8+ T cells as CD4+ T cells.

Embodiment 65. The method of Embodiment 47, wherein between 50 ml and 150 ml of blood are collected.

Embodiment 66. The method of Embodiment 65, wherein the genetically modified T cells and/or NK cells are expanded to a volume between 250 ml and 5 L or between 500 mL and 2.5 L, or between 500 mL and 2 L, or between 1 L and 2 L.

Embodiment 67. The method of Embodiment 65, wherein at least 5 times, 10 times, 15 times, 20 times, 25 times, 30 times, 35 times, 40 times, or 50 times as many genetically modified T cells and/or NK cells are harvested as the number of T cells and/or NK cells that were present in the isolated PBMCs or that were present during the activating step.

Embodiment 68. A method according to any of the method Embodiments provided herein unless recited otherwise, wherein at least 5 times, 10 times, 15 times, 20 times, 25 times, 30 times, 35 times, 40 times, 50 times, 60 times, or 75 times as many cells are harvested as the number of PBMCs that were present in the isolated PBMCs or that were present during the activating step.

Embodiment 69. A method according to any of the method Embodiments provided herein unless recited otherwise, wherein at least 5 times, 10 times, 15 times, 20 times, 25 times, 30 times, 35 times, 40 times, or 50 times, 60 times, or 75 times as many viable cells are harvested as the number of PBMCs that were present in the isolated PBMCs or that were present during the activating step.

Embodiment 70. A method according to any of the method Embodiments provided herein unless recited otherwise, wherein between 5 times and 75 times, or between 10 times and 75 times, or between 20 times and 50 times, or between 25 times and 50 times, as many cells or viable cells are harvested as the number of PBMCs that were present in the isolated PBMCs or that were present during the activating step.

Embodiment 71. The method of Embodiment 65, wherein at least 10 times as many genetically modified T cells are harvested than the number of T cells that were present in the isolated PBMCs or that were present during the activating step.

Embodiment 72. A method according to any of the method Embodiments provided herein unless explicitly recited otherwise, wherein the base media is a commercially available chemically-defined media for ex vivo T-cell cell expansion.

Embodiment 73. The method of Embodiment 72, wherein the cell expansion media further comprises a synthetic sera replacement.

Embodiment 74. The method of Embodiment 72 or 73, wherein the cell expansion media comprises L-glutamine or a dipeptide substitute for L-glutamine

Embodiment 75. The method of any one of Embodiments 72 to 74 wherein the media has the composition of the basal media and media supplement of catalog number A1048501 or A1048503 of Thermo Fisher Scientific.

Embodiment 76. A method according to any of the method Embodiments provided herein unless explicitly recited otherwise, wherein the cell expansion media comprises the composition of the basal media and media supplement of catalog number A1048501 or A1048503 of Thermo Fisher Scientific, further supplemented with L-glutamine or a dipeptide substitute for L-glutamine, a synthetic sera replacement, and IL-2 at a concentration of at least 50 IU/ml.

Embodiment 77. The method of Embodiment 76, wherein the genetically modified T cells are expanded at least 20-fold, 25-fold, 30-fold, 50-fold, 75-fold, 80-fold, 90-fold, 100-fold, or 125-fold.

Embodiment 78. The method of Embodiment 76 or 77, wherein the cell expansion media comprises less than 300 IU/ml of IL-2.

Embodiment 79. The method of Embodiment 76 or 77, wherein the cell expansion media comprises between 50 and 150 IU/ml of IL-2 and a concentration of NAC that is at least 5 mM greater than the concentration of NAC present during the transduction reaction.

Embodiment 80. A method according to any of the method Embodiments provided herein unless explicitly recited otherwise, wherein natural sera is absent during expansion.

Embodiment 81. The method of Embodiment 80, wherein a serum replacement is present during expansion.

Embodiment 82. A method according to any one of methods 76 to 79, or according to any Embodiment herein unless otherwise recited, wherein at least 20 times, or at least 25 times, or between 20 times and 75 times, or between 25 times and 70 times, or between 25 and 50 times, or between 25 and 150 times, or between 50 and 150 times, or between 50 and 135 times as many cells or viable cells are present after the expanding as the number of PBMCs that were present in the isolated PBMCs or that were present during the activating step.

Embodiment 83. A method according to any method Embodiment provided herein unless explicitly stated otherwise, wherein the T cells and/or the NK cells are expanded at least 5-fold, 10-fold, 20-fold, 25-fold, or 50-fold from the number of T cells and/or NK cells in the activation step.

Embodiment 84. A method according to any method Embodiment provided herein unless explicitly stated otherwise, wherein the T cells are expanded at least 5-fold, 10-fold, 20-fold, 25-fold, 50-fold, 75-fold, 100-fold, or 125-fold from the number of T cells and/or NK cells in the activation step.

Embodiment 85. A genetically modified T cell and/or NK produced by a method according to any of the method Embodiments provided herein.

Embodiment 86. A population of genetically modified T cells produced by a method according to any of the method Embodiments provided herein.

Embodiment 87. The population of Embodiment 86, wherein the population has a ratio of at least twice as many CD8 positive cells as CD4 positive cells.

Embodiment 88. A population of any one of Embodiments 86 or 87, wherein the cells are present in a chemically-defined media.

Embodiment 89. A population of Embodiment 88, wherein the cells are present in a media comprising recombinant IL-2.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius (° C.), and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); i.v., intravenous(ly); and the like.

### EXAMPLES

### Example 1

### Ex Vivo Activation, Transduction, and Expansion in a Single Chamber Fed-Batch System

This example successfully demonstrates activation, transduction, and expansion of T cells ex vivo under a number of conditions, including within a single reaction chamber without transferring or washing the cells between any of these steps. Furthermore, this example demonstrates using a fed-batch system wherein media is not exchanged during any step from activation through expansion.

### METHODS

### PBMC Enrichment and Cell Counts

Day 0. Human peripheral blood mononuclear cells (PBMCs) were enriched from a single buffy coat (San Diego Blood Bank) by density gradient centrifugation with Ficoll-Paque PREMIUM® (GE Healthcare Life Sciences) according to the manufacturer's instructions. For cell counting, the red blood cells in the sample were lysed using Red Blood Cell Lysis Solution (BD Biosciences, 555899) according to the manufacturer's instructions. The PBMCs were washed and counted using trypan blue and a hemocytometer.

### Cell Activation

Day 0. The enriched PBMCs were resuspended in 40 ml each of media 1 (M1), media 2 (M2), media 3 (M3), or media 4 (M4) further supplemented with 100 IU/ml recombinant human interleukin-2 (IL-2) (R&D System, 202-IL-500) and 50 ng/ml anti-CD3 antibody (Biolegend, #317326) at 0.5×10⁶ cells/ml. M1 was X-VIVO™ 15 1 L (Lonza). M2 was M1 supplemented with 25 ml CTS™ Immune Cell SR (Thermo Fisher, A2596101). M3 was OpTmizer™ CTS™ T-Cell Expansion Basal Medium 1 L (Thermo Fisher, A10221-01) supplemented with 26 ml OpTmizer™ CTS™ T-Cell Expansion Supplement (Thermo Fisher, A10484-02), and 10 ml CTS™ GlutaMAX™-I Supplement (Thermo Fisher, A1286001). M4 was M3 supplemented with 25 ml CTS™ Immune Cell SR (Thermo Fisher, A2596101). PBMCs resuspended in each of the 4 media were seeded at a concentration of 0.5×10⁶ cells/ml into the wells of a G-Rex 6 Well Plate (Wilson Wolf, 80240M) (3 ml/well PBMCs), a standard 12 well culture plate (Corning, 3513) (1 ml/well PBMCs), or a culture bag (CultiLife 215 Culture bag) (10 ml/bag PBMCs) (Clontech, FU0005) with and without pre-coating with RetroNectin (TakaRa, T100B). The cells were incubated overnight (between 12 and 24 hours) in a standard humidified tissue culture incubator at 37° C. and 5% CO₂.

### Viral Transduction

Day 1. After the overnight incubation, 14.36 μl of a lentivirus particle preparation with a titer of 3.48×10⁸ transduction units/ml was added to each sample for a multiplicity of infection (MOI) of 10. The lentivirus genome encoded an e-TAG. The transduction reaction mixtures were incubated overnight (between 12 and 24 hours) in a standard humidified tissue culture incubator at 37° C. and 5% CO₂.

### T Cell Expansion

Days 2-9. Following the overnight transduction, cells in the standard 12 well culture plates and in the culture bags were transferred into the wells of G-Rex 6 Well Plates. The cells from 3 wells (3 ml total) of the 12 well culture plates were combined into 1 well of the G-Rex 6 Well Plate. The cells from 1 culture bag were split into 3 wells (3 ml/well) of a G-Rex 6 Well Plate. The cells that were transduced in the wells of G-Rex 6 Well Plates were left in these plates. A fed-batch system was tested. Accordingly, the cells were fed by bringing the media volume of each sample to 40 ml using M1, M2, M3, or M4 to match the existing culture media, and adding 100 IU/ml of IL-2. The G-Rex plates were incubated in a standard humidified tissue culture incubator at 37° C. and 5% CO₂ and supplemented beginning on Day 4 with an additional 100 IU/ml IL-2 every 48 hours.

### Harvest, Cell Counts, and Cell Viability

Day 9. PBMCs were collected on Day 9 by gently pipetting the media in each well up and down before collecting each sample into a 50 ml conical tube. The samples were washed and then the cells were counted and analyzed for viability using the Countess II FL Automated Cell Counter (Thermo Fisher, AMQAF1000).

### Flow Cytometry

For each sample, 0.5×10⁶ cells were washed and resuspended in FACS buffer (PBS+2% FBS+0.1% sodium azide). Cells were stained with 100 μl FACS buffer containing 0.9 μg/ml biotinylated-cetuximab for 30 mins on ice. Stained cells were washed with FACS buffer and stained with Streptavidin PE (eBioscience, 12-4317-87, 0.2 mg/ml), CD3-BV421 (Biolegend, 317344), CD4-PE-Dazzle 594 (Biolegend, 300548), and CD8-BV570 (Biolegend, 301038) for 30 mins on ice. Cells were washed twice in FACS buffer, fixed in a 1:1 mixture of the FACS buffer and BD Cytofix (BD #554655), processed with Novocyte (ACEA), and the resulting data was analyzed with NovoExpress software (ACEA) using a lymphocyte gate based on forward and side scatter.

### RESULTS

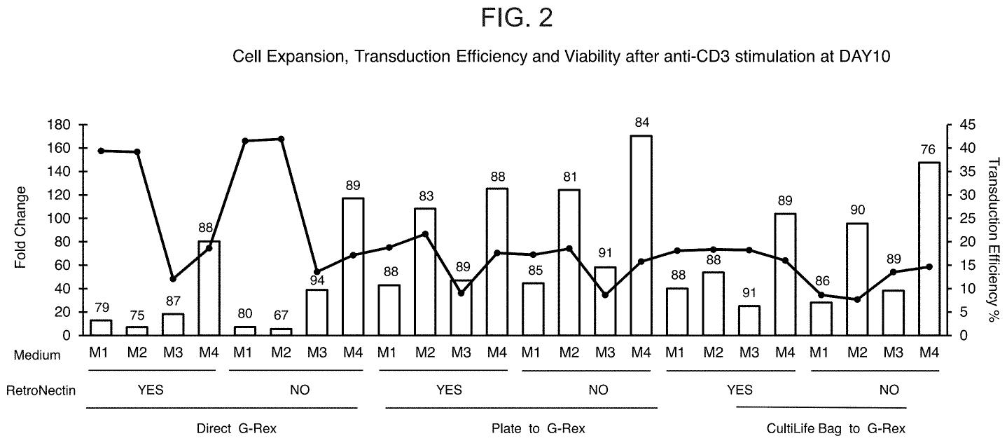

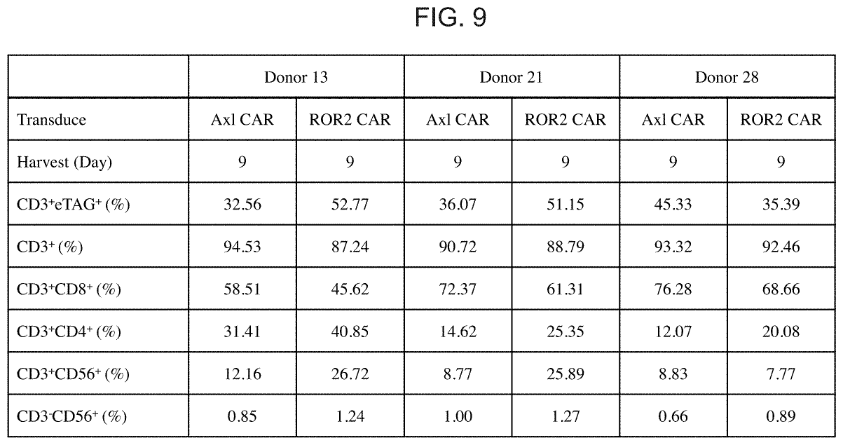

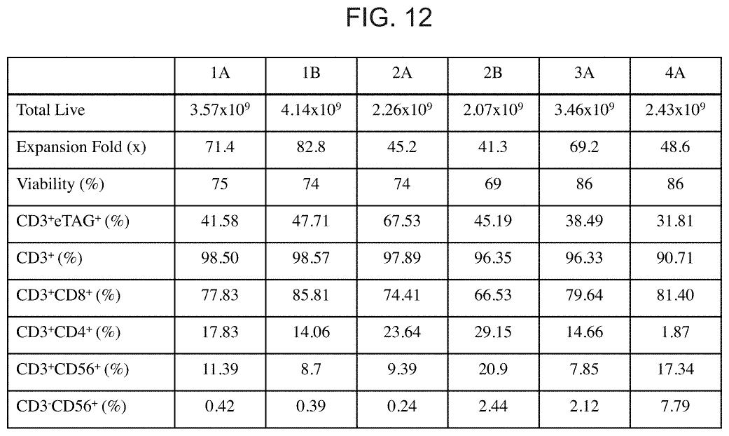

Ex-vivo activation, transduction, and expansion of T cells was tested under a variety of conditions without washing the cells or exchanging media between these steps. Transduction efficiency was over 5% for all conditions tested, and over 40% for some of the samples that remained in the G-Rex for activation and transduction, showing that transduction can be effectively performed within a G-Rex chamber. Later experiments performed under similar conditions achieved transduction efficiencies over 50% (for example, subjects 13 and 21 transduced with a lentivirus particle preparation encoding a CAR directed to ROR2 (ROR2 CAR) in FIG. 9 and sample 2A in FIG. 12). Further unexpectedly, cells from all of the tested conditions were successfully activated, transduced, and expanded even though the cells were not washed between any of these steps (FIG. 2).

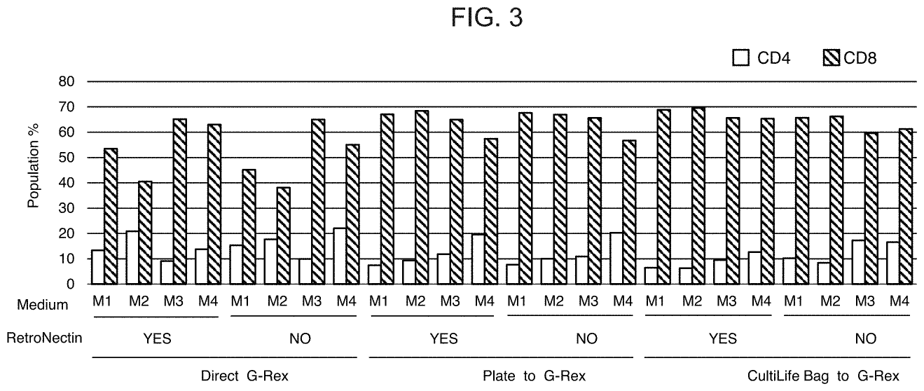

Expansion was performed using a fed-batch method where media and growth factors were added initially, and growth factors were added periodically, but media was not exchanged or perfused. Expansion was successful using the fed-batch method with at least 5-fold expansion based on cell counts between the initial PBMCs in the activation reaction and the number of cells in the expanded cultures at the time of harvest, under all conditions tested and over 50-fold expansion under many of the conditions tested. Some of the largest expansion was obtained with M4 media, OpTmizer™ CTS™ T-Cell Expansion Basal Medium supplemented with OpTmizer™ CTS™ T-Cell Expansion Supplement, CTS™ GlutaMAX™-I Supplement, and CTS™ Immune Cell SR (serum replacement). Furthermore, the addition of a serum replacement appeared to be generally beneficial. The CD8:CD4 phenotype of the cells was skewed toward CD8+ cells (FIG. 3). Greater cell expansion was observed when the cells were activated and transduced in either a plate or a bag and then transferred to a G-Rex (Plate to G-Rex or Culture Bag to G-Rex, respectively). However, such a system requires more handling of the samples and is less amenable to a closed system process than is performing the activation, transduction, and expansion in a single chamber of the G-Rex (Direct to G-Rex). RetroNectin did not significantly affect the transduction of the cells. For cells activated, transduced, and expanded in a single chamber of a G-Rex in M4 media in the absence of RetroNectin, the CD8:CD4 phenotype was 55%:22% or 2.5:1. Greater cell expansion and a CD8:CD4 ratio closer to 1 were observed when the cells were activated using the Dynabeads Human T-Activator CD3/CD28 Kit (Thermo Fisher Scientific), but the beads need to be removed, resulting in more handling of the sample and an increased risk of contamination.

The results reported in this example show that T cells can be activated, transduced, and expanded in a single reaction chamber and never withdrawn from the chamber during the activation, transduction or expansion until they are harvested after expansion. Furthermore, fed-batch processes were successfully demonstrated under a number of different conditions where media was added but not removed or exchanged between the start of activating and completion of expansion. Such fed-batch processes were carried out with 100 IU/ml IL-2. Finally, transduction was successfully performed without RetroNectin and within a G-Rex chamber, which is amenable to a fully closed system.

### Example 2

### Analysis of Various Factors in an Ex Vivo Activation, Transduction, and Expansion Conditions in a Fed-Batch, Single Chamber System

This example demonstrated the effects of IL-2, IL-7, anti-CD28, and supplemental N-acetyl-cysteine (NAC) on the ex vivo activation, transduction, and expansion of T cells within a single reaction chamber in a fed-batch process.

### METHODS

### Blood Collection

Day 0. Whole human blood (100 ml) from a healthy subject was collected into a standard blood collection bag or a tube containing Citrate Phosphate Dextrose (CPD) anticoagulant.

### PBMC Enrichment

Day 0. The blood was processed using density gradient centrifugation with Ficoll-Paque™ (General Electric) using a CS-900.2 kit (BioSafe; 1008) on a Sepax 2 S-100 device (Biosafe; 14000) according to the manufacturer's instructions, to obtain 45 ml of PBMCs. The wash solution used in the Sepax 2 process was Normal Saline (Chenixin Pharm) +2% human serum albumin (HSA) (Sichuan Yuanda Shuyang Pharmaceutical). The final cell resuspension solution was 45 ml Complete OpTmizer™ CTS™ T-Cell Expansion SFM (OpTmizer™ CTS™ T-Cell Expansion Basal Medium 1 L (Thermo Fisher, A10221-03) supplemented with 26 ml OpTmizer™ CTS™ T-Cell Expansion Supplement (Thermo Fisher, A10484-02), 25 ml CTS™ Immune Cell SR (Thermo Fisher, A2596101), and 10 ml CTSTM GlutaMAX™-I Supplement (Thermo Fisher, A1286001)).

### Cell Counts

Day 0. A 0.5 ml aliquot of PBMCs was removed from the final bag of the CS-900.2 kit using a 1 ml syringe connected to the luer port. Prior to performing a cell count, red blood cells in each aliquot were lysed using Red Blood Cell Lysis Solution (BD Biosciences, 555899) according to the manufacturer's instructions. The remaining cells were counted and analyzed for viability using the Countess II FL Automated Cell Counter.

### Cell Activation

Day 0. 1.5×10⁶ viable PBMCs in 3 ml of Complete OpTmizer™ CTS™ T-Cell Expansion SFM (media M4 as described in Example 1) were aseptically seeded into the wells of G-Rex 6 Well Plates. Anti-CD3 antibody (OKT3, Novoprotein) was added to all the samples to a final concentration of 50 ng/ml. IL-2 (Novoprotein) was added to samples 1, 2A, 2B, 3, 5, 6A, and 6B to a final concentration of 100 IU/ml. IL-2 was added to sample 4 to a final concentration of 300 IU/ml. IL-7 (Novoprotein) was added to samples 3, 6A, and 6B to a final concentration of 10 ng/ml. Anti-CD28 antibody (Novoprotein) was added to sample 5 to a final concentration of 50 ng/ml. A sufficient amount of NAC (Sigma) was added to samples 2A and 6A to increase the concentration of NAC by 10 mM. The G-Rex plates were incubated overnight (between 12 and 24 hours) in a standard humidified tissue culture incubator at 37° C. and 5% CO₂.

### Viral Transduction

Day 1. After overnight incubation, 125 μl of a lentivirus particle preparation was added to each sample at a multiplicity of infection (MOI) of 5. The lentivirus genome encoded an anti-Ax1 MRB-CAR that included an ASTR, a stalk, a transmembrane domain, and an intracellular domain, and a co-stimulatory domain, and expressed eTag on the same transcript. The transduction reaction mixtures were incubated overnight (between 12 and 24 hours) in a standard humidified tissue culture incubator at 37° C. and 5% CO₂.

### T Cell Expansion

Days 2-11. Following the overnight incubation, the cells were fed by bringing the total volume in each well of the G-Rex 6 Well Plates to 30 ml with Complete OpTmizer™ CTS™ T-Cell Expansion SFM (Media 4 of Example 1). Samples 2A and 6A, which contained NAC on Day 0 were supplemented with a sufficient amount of NAC to keep the final concentration of NAC at 10 mM. NAC was also added to samples 2B and 6B, which had not received NAC previously, to a final concentration of 10 mM NAC. Additional cytokine was fed to the cells on Day 2 and every 48 hours as follows; 100 IU/ml of IL-2 (Novoprotein) was added to samples 1, 2A, 2B, 3, 5, 6A and 6B, 300 IU/ml of IL-2 was added to sample 4, and 10 ng/ml IL-7 (Novoprotein) was added to samples 3, 6A, and 6B.

### Harvest, Cell Counts, and Cell Viability

Day 11. PBMCs were collected on Day 11 by gently pipetting the media in each well up and down before collecting each sample into a 50 ml conical tube. The samples were washed and then the cells were counted and analyzed for viability using the Countess II FL Automated Cell Counter. The cells were frozen at a final density of 1×10⁷ cells/ml in RPMI-1640 (Gibco) supplemented with 20% heat-inactivated FBS (Gibco) and 10% dimethylsulfoxide using a Mr. Frosty Freezing Container (Thermo Fisher, or a CoolCell Cell Freezing Container (BioCision) according to the manufacturers' instructions. Cryopreserved cells were stored in liquid nitrogen for future use.

### Flow Cytometry

Cryopreserved cells harvested on Day 11 were thawed and 0.5×10⁶ cells per staining condition were resuspended in FACS buffer (PBS+2% FBS+0.1% sodium azide). Cells were stained with 100 μl FACS buffer containing biotinylated-cetuximab (cetuximab was obtained from Chembest (Catalog # C13458) and biotinylated by BioDuro (San Diego, Calif.)) for 30 mins on ice. Stained cells were washed with FACS buffer and stained with Streptavidin PE (eBioscience, 12-4317-87, 0.2 mg/ml) for 30 mins on ice. Cells were washed twice in FACS buffer, fixed in a 1:1 mixture of the FACS buffer and BD Cytofix (BD Biosciences, 554655), processed with Novocyte (ACEA), and the resulting data was analyzed with NovoExpress software (ACEA) using a lymphocyte gate based on forward and side scatter. Successful transduction of T cells was measured as the percentage of CD3+eTAG+ cells.

### RESULTS

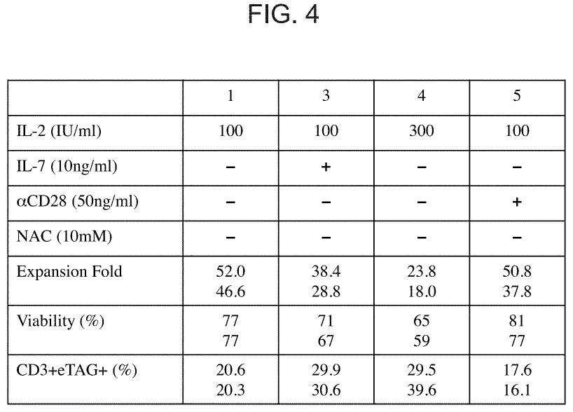

T cells were successfully activated, transduced, and expanded ex vivo within a single reaction chamber of a G-Rex under various conditions. FIG. 4 shows the expansion fold, percent viability, and the transduction efficiency (percentage of CD3+eTAG+ cells) for various conditions. IL-2 was tested at concentrations of 100 or 300 IU/ml IL-2 in the media. The effects of 10 ng/ml IL-7 or 50 ng/ml anti-CD28 were compared to samples lacking both. Cells supplemented with NAC at Day 0 or Day 2 were compared to a sample without supplemented NAC. Both additional IL-2 (300 IU/ml IL-2; FIG. 4 sample 4) and the addition of IL-7 (10 ng/ml IL-7; FIG. 4 sample 3) increased the transduction efficiency (i.e. percentage of CD3+eTAG+ cells), but reduced the expansion fold and percent viability relative to a sample that had 100 IU/ml IL-2 and no IL-7 (FIG. 4, sample 1). Not to be limited by theory, it still may be an advantage to include IL-7 because it is believed that including IL-7 results in more T cells having a less differentiated phenotype, which may be helpful during expansion after the transduced PBMCs are introduced into a subject (Xu et al., Blood (2014) 123:3750-59). A similar experiment demonstrated 50 IU/ml IL-2 was effective in that transduced T cells were expanded and harvested, although 100 IU/ml IL-2 provided better results (data not shown). The addition of anti-CD28 did not significantly affect the expansion fold, percent viability, or transduction efficiency (i.e. percentage of CD3+eTAG+ cells) (see samples 1 and 5 in FIG. 4).

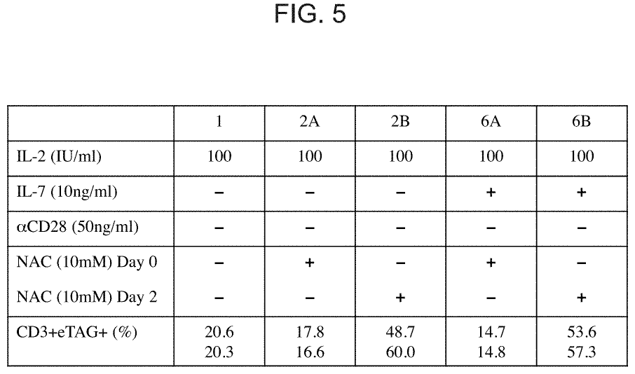

FIG. 5 shows the results focused on the addition of NAC at different times during the process. Additional NAC at Day 0 (before transduction) reduced the percentage of CD3+eTAG+ cells in the absence (sample 2A) or presence (sample 6A) of 10 ng/ml IL-7 relative to a sample that had NAC only present in the commercial media and no IL-7 (sample 1). Surprisingly, additional NAC at Day 2 (after transduction) greatly increased the percentage of transduced cells in the absence (sample 2B) or presence (sample 6B) of 10 ng/ml IL-7 relative to the sample that had no supplemental NAC or IL-7 (sample 1).

These results demonstrate an effective and simple method of activating, transducing, and expanding T cells ex vivo, within a single chamber in a fed-batch manner, where supplemental NAC is added after transduction, IL-7 and anti-CD28 are optional, and no washes are performed during the activating, transducing, or expanding steps. IL-2 at a concentration of 100 IU/ml provided the best results in the system tested. IL-7 and anti-CD28 were optional as neither significantly affected the results. Surprisingly, supplemental NAC had an inhibitory effect when added at Day 0 and a stimulatory effect when added at Day 2.

### Example 3

### Further Characterization of Ex Vivo Activation, Transduction, and Expansion in a Fed-Batch, Single Chamber System

The following example further demonstrates and characterizes activation, transduction, and expansion in a fed-batch, single chamber system. Furthermore, the example analyzes lactate concentration as a surrogate for cell density.

### METHODS

### Blood Collection

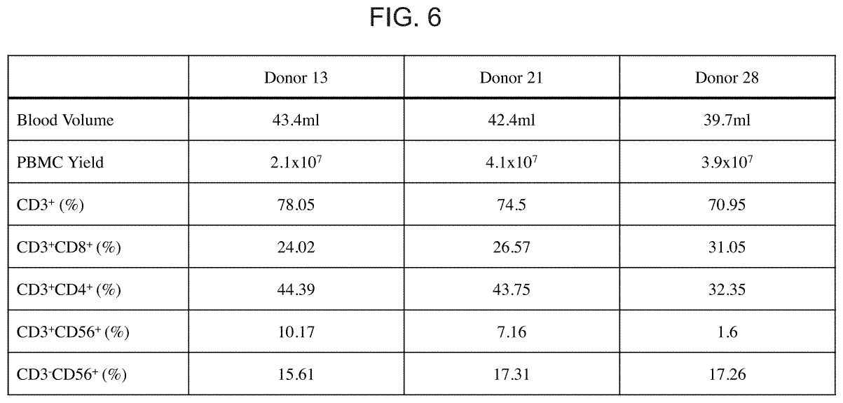

Day 0. Whole human blood (˜40 ml) from each of 3 healthy subjects (Subjects 13, 21, and 28) was collected into a standard blood collection bag or a tube containing Citrate Phosphate Dextrose (CPD) anticoagulant. The volume of blood collected from each subject is shown in FIG. 6. The methods were not all performed on the same day for the different subjects.

### PBMC Enrichment

Day 0. Each sample of blood was processed within 6 hours of collection as in Example 2.

### Cell Counts

Day 0. A 0.5 ml aliquot of PBMCs from each sample was removed and the red blood cells in that aliquot were lysed prior to cell counting as in Example 2. The total PBMC yield from each subject is shown in FIG. 6. 2×10⁶ PBMCs from each sample were removed and frozen according to the same protocol of Example 2. Cryopreserved cells were stored in liquid nitrogen for later analysis by FACS.

### Cell Activation

Day 0. Replicates of 1.5×10⁶ viable PBMCs from each subject were aseptically seeded in each well of G-Rex 6 Well Plates and the volumes were brought to 3 ml (5×10⁵ viable PBMC cells/ml) with Complete OpTmizer™ CTS™ T-Cell Expansion SFM (media M4 as described in Example 1) supplemented with 100 IU/ml recombinant human interleukin-2 (IL-2) (Novoprotein) and 10 ng/ml recombinant human interleukin-7 (IL-7) (Novoprotein). PBMCs from Subject 13 were seeded in triplicate, PBMCs from Subject 21 were seeded in quadruplicate, and PBMCs from Subject 28 were seeded in quadruplicate 50 ng/ml anti-CD3 antibody (OKT3, Novoprotein) was added to each well to activate the PBMCs for viral transduction. No NAC was added to any of the samples. The G-Rex plates were incubated overnight (between 12 and 24 hours) in a standard humidified tissue culture incubator at 37° C. and 5% CO₂.

### Viral Transduction

Day 1. After overnight incubation, either 125 μl of a lentivirus particle preparation encoding an MRB-CAR directed to Ax1 (Ax1 CAR) or 23 μl of a lentivirus particle preparation encoding an MRB-CAR directed to Ror2 (Ror2 CAR), was added to each well at a multiplicity of infection (MOI) of 5 to form a transduction reaction mixture. The lentivirus genomes encoded CARs that included an ASTR, a stalk, a transmembrane domain, an intracellular domain, and a co-stimulatory domain, and expressed an e-TAG on the same transcript. The transduction reaction mixtures were incubated overnight (between 12 and 24 hours) in a standard humidified tissue culture incubator at 37° C. and 5% CO₂.

### T Cell Expansion

Day 2. Following the transduction reaction overnight incubation, the cells were fed by bringing the total volume of each well of the G-Rex 6 Well Plates to 30 ml with Complete OpTmizer™ CTS™ T-Cell Expansion SFM (Media 4 of Example 1) containing 10 mM NAC. Additionally, 100 IU/ml of IL-2 (Novoprotein) and 10 ng/ml IL-7 (Novoprotein) were added to each well on Day 2 and every 48 hours thereafter.

### Measurement of Lactate and Glucose

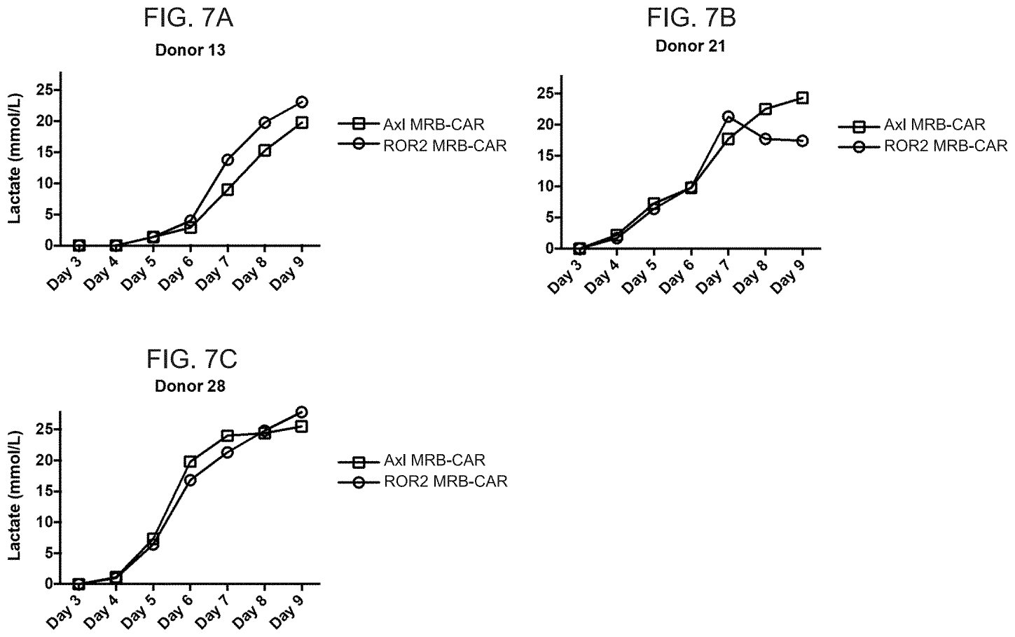

The concentration of lactate in the media was measured daily for each well for Subjects 13 and 21, and for an equal mixture from each well for Subject 28, beginning on Day 3 (FIG. 7). Lactate was measured using a Lactate Plus meter (Nova Biomedical). The concentrations of glucose in the media of each well was also measured daily beginning on Day 3. Glucose was measured using an Accu-Chek Aviva Plus meter (Roche) or an Accu-Chek Performa meter (Roche). The expanded cells from Subjects 13, 21, and 28 were harvested on Day 9. Decreases in glucose concentrations correlated with increases in lactate concentrations, indicating that the cells were not contaminated with bacteria during the expansion.

### Harvest, Cell Counts, and Cell Viability

Expanded PBMCs were harvested on Day 9 by gently pipetting the media in each well up and down before collecting each sample into a 50 ml conical tube. The samples were washed and then the cells were counted and analyzed for viability using the Countess II FL Automated Cell Counter. The cells were frozen according to the same protocol of Example 2. Cryopreserved cells were stored in liquid nitrogen for analysis by FACS.

### Flow Cytometry

Cryopreserved cells were thawed and 0.5×10⁶ cells for each staining condition were washed and resuspended in FACS buffer (PBS+2% FBS+0.1% sodium azide). Cells were stained with 100 μl FACS buffer containing biotinylated-cetuximab for 30 mins on ice. Stained cells were washed with FACS buffer and stained with a cocktail of either Streptavidin FITC (Becton Dickenson, 554060), CD3-PerCp-Cy5.5 (Becton Dickenson, 560835), CD4-APC (Becton Dickenson, 551980), and CD8-PE (Becton Dickenson, 557086), or Streptavidin FITC (Becton Dickenson, 554060), CD3- PerCp-Cy5.5 (Becton Dickenson, 560835), and CD56-PE (Becton Dickenson, 555516) for 30 mins on ice. Cells were washed twice in FACS buffer, fixed in a 1:1 mixture of the FACS buffer and BD Cytofix (BD Biosciences, 554655), processed with Novocyte (ACEA), and the resulting data was analyzed with NovoExpress software (ACEA) using a lymphocyte gate based on forward and side scatter.

### RESULTS

The methods established in the experiments provided in Example 2 for activating, transducing, and expanding T cells ex vivo without washing or transferring the T cells during these steps and using a fed-batch process in systems that are amenable to being fully closed, were tested on blood samples collected from 3 different healthy human subjects. Blood from the healthy subjects was collected in bags and initially processed in a Sepax device to enrich and wash the PBMCs. Approximately 40 ml of blood was used for processing, which yielded between 2.1×10⁷ and 4.1×10⁷ PBMCs, between 71 and 78% of which were T cells (CD3⁺) and 16 to 17% NK cells (CD3⁻CD56⁺) (FIG. 6).

For activation, 1.5×10⁶ viable PBMCs were then seeded in each well of G-Rex 6 Well Plates and the volumes were brought to 3 ml (5×10⁵ viable PBMC cells/ml) with Complete OpTmizer™ CTS™ T-Cell Expansion SFM (media M4 as described in Example 1) supplemented with 100 IU/ml recombinant IL-2 and 10 ng/ml recombinant IL-7. Next, 50 ng/ml of anti-CD3 antibody was added to the PBMCs to form activation reaction mixtures, and the cells were incubated overnight to activate the T cells within the enriched PBMCs. Following activation, one of two lentivirus particle preparations (Ror2 CAR and Ax1 CAR) encoding either of two CARs were added directly to each sample within the wells, at a multiplicity of infection (MOI) of 5, without washing the cells between the activation and the transduction. The cells were incubated until the following day in the transduction reaction mixture.

Following transduction, cells were expanded without washing or transferring the cells between the transduction and the expansion. For expansion, the transduced T cells were fed by bringing the total volume of each well of the G-Rex 6 Well Plates to 30 ml with Complete OpTmizer™ CTS™ T-Cell Expansion SFM (Media 4 of Example 1) containing 10 mM NAC. Additionally, 100 IU/ml of IL-2 (Novoprotein) and 10 ng/ml IL-7 (Novoprotein) were added to each well on Day 2 and every 48 hours thereafter. The cells were allowed to expand up to Day 9 from the original blood collection (Day 0).

Lactate concentrations were measured throughout expansion (FIG. 7). The change of lactate concentration followed a lag phase (continuing through Day 4 or Day 5, depending on the subject), a log phase and a plateau phase (starting at between about Day 6 and Day 9, depending on the subject), as would be expected for changes in cell density during cell expansion. In fact, in other experiments, a correlation between lactate concentration and cell density was confirmed (data not shown). Lactate concentrations appeared to plateau after a concentration of around 20 nmol/L was reached.

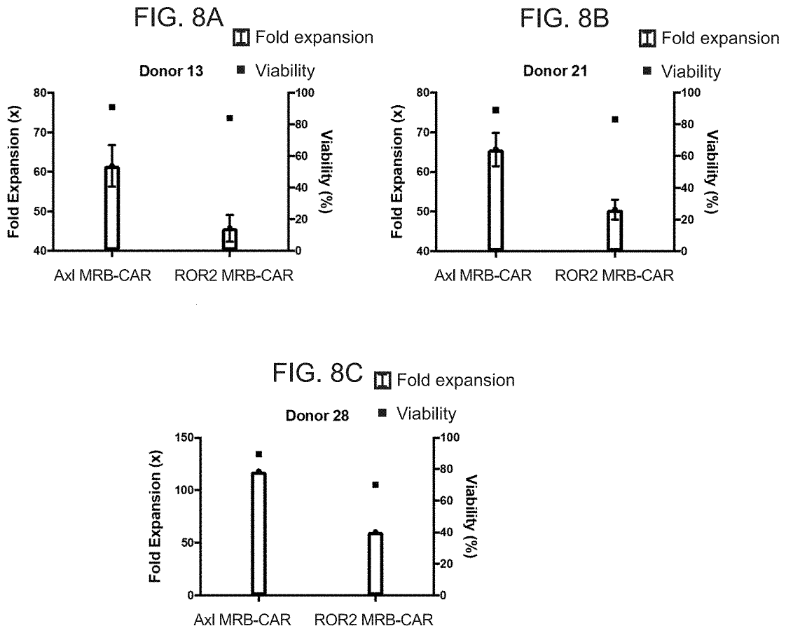

The expanded cells were harvested on Day 9 and analyzed. Cell counts revealed that the T cells were expanded over 40-fold (between 45-fold and 118-fold) for all samples, confirming the effectiveness of the activation, transduction, and expansion process (FIG. 8). Furthermore, cell viability was over 70% for all samples (FIG. 8) Immune cell marker analysis of the expanded population of cells was performed as shown in FIG. 9. Between 32% and 53% of the expanded cells were transduced T-cells that expressed an eTAG (i.e. genetically modified T cells). Between 45% and 77% of the expanded cells were CD8+ T cells and between 12% and 41% were CD4+ T cells. Between 7% and 27% of the expanded cells were NK T cells. Between 0.6 and 1.3% of the cells were NK cells.

Taken together, these results, confirm that the disclosed method for activating, transducing, and expanding T cells ex vivo without washing or transferring the T cells during these steps, using a fed-batch process in systems that are amenable to being fully closed, is effective for transducing and expanding T cells. The potential for the systems utilized to be within a fully closed system provides an opportunity to reduce possible contamination during ex vivo processing of T cells.

### Example 4

### Full Scale Ex Vivo Activation, Transduction, and Expansion in a Fed Batch in a Single Chamber of a Closed System

This example demonstrates a full scale 1 L single chamber, fed-batch closed system for ex vivo expansion of CAR-T cells using the conditions established in Examples 1 and 2, i.e. 100 IU/ml IL-2, 10 ng/ml IL-7, and 50 ng/ml anti-CD3 antibody during activation, and 10 mM supplemental N-acetyl-cysteine (NAC) added after transduction.

### METHODS

### Blood Collection

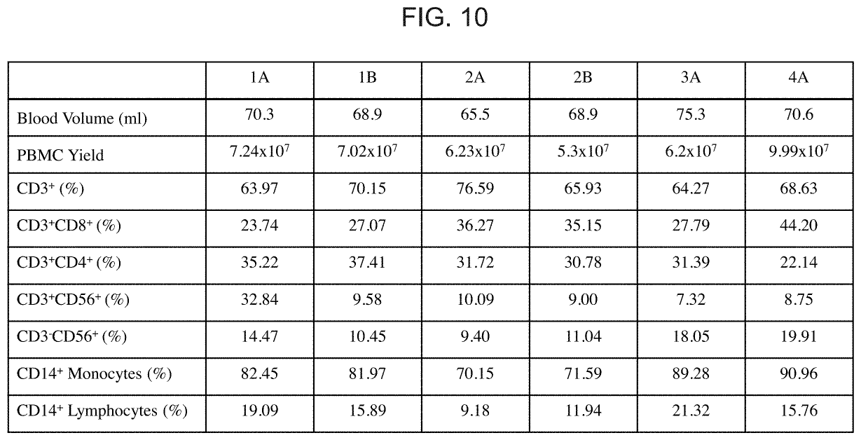

Day 0. Whole human blood from each of 4 healthy subjects was collected into multiple 100 mm Vacutainer tubes (Becton Dickenson; 364606) containing 1.5 ml of Acid Citrate Dextrose Solution A anticoagulant. For each subject, blood from the Vacutainer tubes was pooled and distributed to 2 standard 500 ml blood collection bags for processing separately. The total blood volume for each pooled sample that was processed is shown in FIG. 10 for subjects 1-4 with different pooled samples for a subject designated as “A” or “B.”

### PBMC Enrichment

Day 0. Each sample of blood was processed within 6 hours of collection within a closed system that included a Sepax unit as described in Example 2.

### Cell Counts

Day 0. A 0.5 ml aliquot of PBMCs from each sample was removed and the red blood cells in that aliquot were lysed prior to cell counting as performed in Example 2. 5.0×10⁶ viable Day 0 cells were removed by syringe from a sterile port on the output bag from the Sepax unit and frozen according to the protocol in Example 2. Cryopreserved cells were stored in liquid nitrogen for later analysis by FACS.

### Cell Activation

Day 0. 5.0×10⁷ viable PBMCs were aseptically transferred from each blood collection bag to a reaction chamber of a 1 L G-Rex closed cell culture system (Wilson-Wolf 100M CS) using sterile ports and tubing. The volume was brought to 100 ml (5×10⁵ viable PBMC cells/ml) with Complete OpTmizer™ CTS™ T-Cell Expansion SFM (Media 4 of Example 1) and supplemented with 100 IU/ml recombinant human interleukin-2 (IL-2) (Novoprotein), 10 ng/ml recombinant human interleukin-7 (IL-7) (Novoprotein), and 50 ng/ml anti-CD3 antibody (OKT3, Novoprotein) to activate the PBMC for viral transduction. The G-Rex devices were incubated overnight (between 12 and 24 hours) in a standard humidified tissue culture incubator at 37° C. and 5% CO₂ to activate T cells.

### Viral Transduction

Day 1. After the overnight incubation, 506 μl of a lentivirus particle preparation was added to the reaction chamber of the G-Rex device at a multiplicity of infection (MOI) of 2.5. The lentivirus genome encoded a CAR that included an ASTR, a stalk, a transmembrane domain, an intracellular domain, and a co-stimulatory domain, and expressed an e-TAG on the same transcript. The G-Rex device was incubated overnight (between 12 and 24 hours) in a standard humidified tissue culture incubator at 37° C. and 5% CO₂.

### T Cell Expansion

Days 2-12. Following the overnight incubation, the cells were fed by bringing the total volume in the chamber of each G-Rex device to 1 L with Complete OpTmizer™ CTS™ T-Cell Expansion SFM (Media 4 of Example 1) supplemented with a sufficient amount of NAC (Sigma) to result in a final concentration of 10 mM NAC along with 100 IU/ml recombinant human IL-2 and 10 ng/ml recombinant human IL-7. The G-Rex device was incubated in a standard humidified tissue culture incubator at 37° C. and 5% CO2 with additions of 100 IU/ml recombinant human IL-2 and 10 ng/ml recombinant human IL-7 solution every 48 hours.

### Measurement of Lactate and Glucose

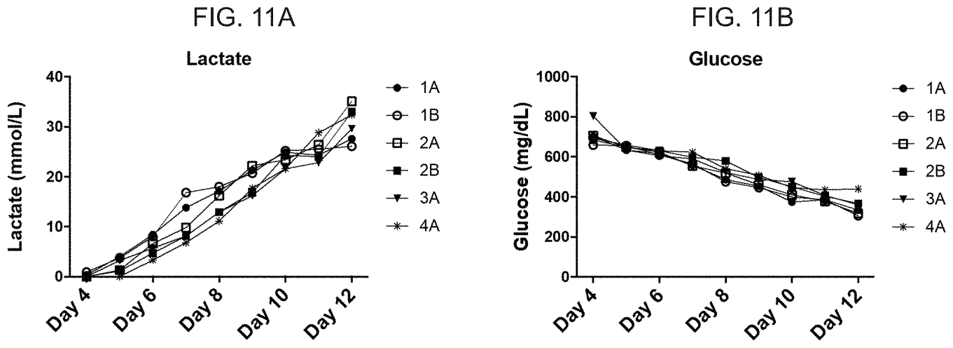

The concentration of lactate in the media of each well was measured daily beginning on Day 4 (FIG. 11). The lactate concentrations were measured according to the manufacturer's instructions using either a Lactate Plus meter (Nova Biomedical) or a biochemistry analyzer (YSI). The glucose concentrations were measured according to the manufacturer's instructions using an Accu-Chek Aviva Plus meter (Roche), an Accu-Chek Performa meter (Roche), or a biochemistry analyzer (YSI). The decreases in glucose concentrations correlated with increases in lactate concentrations, indicating that the PMBCs were not contaminated with bacteria during the expansion.

### Harvest, Cell Counts, and Cell Viability

On Day 12, a manual process using a pipet, or an automated process using a GatheRex device (Wilson Wolf) was used to remove excess media from the top of the G-Rex closed cell culture system according to the manufacturer's instructions. Following media removal, a pipet or the GatheRex device was used to transfer the concentrated cell product into a 500 ml IV bag. A cell count was taken for the cell product in the IV bag using Trypan blue and a Countess device (Thermo Fisher). The harvested cells were washed and concentrated using a CS900.2 kit (BioSafe; 1008) on a Sepax 2 S-100 device (Biosafe; 14000) following the manufacturer's instructions using three cell wash cycles and a final volume selected to result in 1×10⁸ viable cells/ml. The wash solution used in the Sepax 2 process was Normal Saline plus 2% human serum albumin (HSA). The final cell product resuspension solution was 5% glucose in normal saline (DSNS, Shandong Qidu) plus 2% HSA plus 20 g/L Sodium Bicarbonate (NaHCO₃) (Shanghai Experiment Reagent Co.). The PBMCs were frozen according to the protocol in Example 2. Cryopreserved cells were stored in liquid nitrogen.

### Flow Cytometry

Aliquots of the cryopreserved PBMCs from Day 0 and Day 12 were quickly thawed in a 37° C. water bath. The thawed PBMCs were then washed and resuspended in Stain Buffer (BD Biosciences, 554656) and incubated with Human Fc block (Becton Dickenson) for 10 min on ice. PBMCs from Day 0 were stained for 30 minutes on ice with 50 μl FACS buffer containing 2.5 μl of each of the following antibodies; CD3-BV421 (Biolegend), CD8-BV510 (Biolegend), CD4-PE-Cy7 (Biolegend), CD56-BV785 (Biolegend), and CD14-PE (Biolegend) for 5 color staining. PBMCs from Day 12 were stained the same way except that the anti CD-14-PE antibody was not included in the staining cocktail. Cells were washed twice in FACS buffer, fixed in a 1:1 mixture of FACS buffer and BD Cytofix (BD Biosciences, 554655), processed with Novocyte (ACEA), and the resulting data was analyzed with NovoExpress software using a lymphocyte gate or in the case indicated in FIG. 10 for CD14, a monocyte gate, based on forward and side scatter.

### RESULTS

A large-scale closed system that included a G-Rex unit was used to activate, transduce, and expand CAR-T cells ex-vivo without washing or transferring the cells during or between these steps, within a single reaction chamber of the G-Rex under fed-batch conditions. Blood was collected from healthy subjects. PBMCs were enriched within a closed Sepax unit. The enriched PBMCs were analyzed (FIG. 10). Between 64 and 77% of the collected PBMCs were T cells. The ratios of CD8 to CD4 cells in the isolated, enriched population varied between about 1:1 and about 2:1 from sample to sample. Between 9 and 22% of the PBMCs that fell within the lymphocyte gate were CD14 positive lymphocytes and between 70 and 91% of the PBMCs that fell within the monocyte gate were CD14 positive monocytes or macrophages. Not to be limited by theory, it is believed that the presence of antigen presenting cells potentially assists in the activation process by presenting the anti-CD3 along with endogenously expressed coreceptors.

5×10⁷ viable PBMCs of the isolated, enriched PBMCs were transferred from the Sepax unit using sterile tubing into the reaction chamber of a G-Rex system. T cells within the PBMCs were activated by overnight incubation in a reaction mixture that contained soluble anti-CD3 antibody in a T cell expansion media. The activated cells were then transduced by adding a lentivirus encoding a CAR to the activation reaction mixture and incubating until the following day, without washing the cells or exchanging the media between the activation and the transduction.

The transduced T cells were then expanded in T cell expansion media with supplemental NAC, all within the same reaction chamber of the G-Rex using conditions established in small-scale (See Examples 1-3). No washing or transferring of the transduced cells was performed between the transduction and the expansion. The media used for the activation, transduction, and expansion was Complete OpTmizer CTS T-Cell Expansion serum free media supplemented with 100 IU/ml recombinant human interleukin-2 (IL-2) and 10 ng/ml recombinant human interleukin-7 (IL-7). Cell expansion was performed within the reaction chamber using a fed-batch method where the media volume was increased from about 100 ml to about 1 L with the T cell expansion media, and NAC was added to the T cell expansion media to a final concentration of 10 mM NAC. 100 IU/ml recombinant human IL-2 and 10 ng/ml recombinant human IL-7 were present in the cell expansion media during activation and transduction, and added into the cell expansion media every 48 hours during expansion. No additional NAC was added to the activation and transduction reaction mixtures beyond any NAC present in the OpTmizer media. Expansion was carried out up to 12 days after the start of activation. Media was not exchanged during the activation, transduction, or expansion and the activated T cells remained in the reaction chamber for these steps.

FIG. 11 shows lactate and glucose concentrations during cell expansion using this large-scale, fed-batch method. As discussed in Example 3, lactate levels provide a surrogate measurement for cell densities. Lactate levels continued to increase to Day 12 to concentrations over 20 nmol/L. Furthermore, the decreases in glucose concentrations correlated with increases in lactate concentrations, suggesting that there were no bacterial contaminations during the expansion.

FIG. 12 provides the results of cellular analysis of the expanded cells upon harvest at Day 12. Between 2.0×10⁹ and 4.2×10⁹ viable cells were harvested from the expanded cells and between 69 and 86% of the expanded cells were viable. This represented an expansion of between 41-fold and 83-fold. Of the harvested cells, between 90% and 99% of the harvested cells were T cells. The ratio of CD8:CD4 T cells was over 2 for all samples. Between 7% and 21% of the expanded cells were NK T cells. Between 0.24% and 7.79% of the expanded cells were NK cells.

These results establish the effectiveness of the disclosed large-scale, closed method where activation, transduction, and expansion were performed within a single chamber of a closed system, without washes or media exchanges, in a fed-batch process. Such a process offers advantages of simplicity, robustness, less chance of contamination, and cost savings, which make this method amenable to more widespread adoption than current methods.

### Example 5

### Ex Vivo Activation, Transduction, and Expansion in a Clinical Setting

This example provides details of a full-scale method within a closed system that can be used for preparing CAR-T cells for reintroduction into a subject, wherein the activation, transduction, and expansion are performed within a single reaction chamber of the closed system, without washing between these steps and using a fed-batch protocol for cell expansion. The method utilizes 100 IU/ml IL-2, 10 ng/ml IL-7, and 50 ng/ml anti-CD3 antibody added before transduction, and 10 mM N-acetyl-cysteine (NAC) added after transduction.

### EXEMPLARY LARGE-SCALE METHODS

### Closed System

The system for performing T cell processing provided in this example is closed from blood collection through harvesting. The system includes a Sepax device, a G-Rex device, and a GatheRex device. The blood collection bag, Sepax device, G-Rex device, and GatheRex device are connected using sterile welded connections and sterile tubing sets. Media is purchased in sterile bags with self-healing ports.

### Blood Collection

Whole human blood (80-100 ml) is collected into a standard blood collection bag containing an anticoagulant, either Acid Citrate Dextrose (ACD) or Citrate Phosphate Dextrose (CPD).

### PBMC Enrichment

The blood is processed within 6 hours of collection using density gradient centrifugation with Ficoll-Paque™ (General Electric) using a CS900.2 (BioSafe; 1008) kit on a Sepax 2 S-100 device (Biosafe; 14000) according to the manufacturer's instruction, to enrich peripheral blood mononuclear cells (PBMCs). The PBMCs are washed with two wash cycles of 45 ml volume. The wash solution in the Sepax 2 process is normal saline plus 2% human serum albumin (HSA) (Sichuan Yuanda Shuyang Pharmaceutical Co., Ltd). The final cell resuspension solution is about 45 ml of a media made by supplementing 1 L of Complete OpTmizer CTS T-Cell Expansion SFM (OpTmizer CTS T-Cell Expansion Basal Medium (Thermo Fisher, A10221-03) with 26 ml OpTmizer CTS T-Cell Expansion Supplement (Thermo Fisher, A10484-02), 25 ml CTS Immune Cell SR (Thermo Fisher, A2596101), and 10 ml CTS™ GlutaMAX™-I Supplement (Thermo Fisher, A1286001)).

### Cell Activation

A cell count is performed on the enriched PBMCs using a Nucleocounter NC200 device (Chemometec). 5×10⁷ viable PBMC cells are transferred through sterile ports and tubing to a 1 L G-Rex closed cell culture system (Wilson-Wolf, 100M CS) at 5×10⁵ viable PBMC cells/ml in Complete OpTmizer CTS T-Cell Expansion SFM with 100 IU/ml recombinant human interleukin-2 (IL-2) (Novoprotein), 10 ng/ml recombinant human interleukin-7 (IL-7) (Novoprotein), and 50 ng/ml anti-CD3 antibody (OKT3, Novoprotein) to activate the PBMCs for viral transduction. Thus, typically the volume of the activation reaction mixture is 100 ml. In the case that fewer than 5×10⁷ viable PBMC cells are enriched, the total number of viable PBMCs added to the G-Rex is reduced to the total amount enriched keeping the concentration of cells in the G-Rex device constant at 5×10⁵ viable PBMCs/ml. The G-Rex device is incubated overnight (between 12 and 24 hours) in a standard humidified tissue culture incubator at 37° C. and 5% CO₂.

### Viral Transduction

After overnight incubation, a lentivirus particle preparation encoding a CAR, for example an MRB-CAR, is added to the G-Rex device at a multiplicity of infection (MOI) of 2.5. The G-Rex device is incubated overnight (between 12 and 24 hours) in a standard humidified tissue culture incubator at 37° C. and 5% CO₂.

### T Cell Expansion

Following the overnight incubation, the total volume in the G-Rex device is brought to 1 L with Complete OpTmizer CTS T-Cell Expansion SFM supplemented with a sufficient amount of NAC (Sigma) to result in a final concentration of 10 mM NAC in the G-Rex device along with 100 IU/ml recombinant human IL-2 and 10 ng/ml recombinant human IL-7. The G-Rex device is incubated in a standard humidified tissue culture incubator at 37° C. and 5% CO₂ with additions of 100 IU/ml recombinant human IL-2 and 10 ng/ml recombinant human IL-7 solution every 48 hours by injection from a syringe through a sterile port on the G-Rex device. Glucose and lactate levels are tested daily by withdrawing 1 ml samples and analyzing the samples using a biochemistry analyzer (YSI). This process is continued up to 12 days.

### Harvest and Cell Counts

On the day of the expanded cell product harvest, a GatheRex device (Wilson Wolf) is used to reduce the cell volume containing the cells in the G-Rex device by removing excess media from the top of the G-Rex device according to the manufacturer's instructions. After media removal, the GatheRex device is used to transfer the concentrated cell product into a 500 ml IV bag. A cell count is taken for the cell product in the IV bag using a Nucleocounter NC-200 device. The cell product is washed and concentrated using a CS900.2 (BioSafe; 1008) kit on a Sepax 2 S-100 device (Biosafe; 14000) following the manufacturer's instructions with three cell wash cycles, and the final volume selected to result in 1×10⁸ viable cells/ml. The wash solution and final cell product resuspension solution used in the Sepax 2 process is D5NS (Shandong Qidu) plus 2% HSA (Sichuan Yuanda Shuyang Pharmaceutical Co., Ltd) plus 20 g/L Sodium Bicarbonate (NaHCO₃) (Shanghai Experiment Reagent Co,. Ltd or Dongya Pharmaceutical Co., Ltd). Cells can optionally be cryopreserved according to the protocol in Example 2.

### RESULTS

The above methods were performed except the blood collection was performed using tubes. PBMCs were successfully transduced using this large-scale, closed system method. Furthermore, 95-fold expansion was achieved based on cell counts of total cells after expansion at day 10(3.66×10⁹ cells) versus the number of PBMCs in the activation reaction (3.86×10⁷ cells). In follow-on experiments between 40 and 134-fold expansion was achieved, with numerous runs exceeding 100-fold expansion.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

What is claimed is:

1. A method for transducing T cells and/or NK cells from isolated blood, comprising:
a) enriching peripheral blood mononuclear cells (PBMCs) to isolate PBMCs comprising T cells and/or NK cells from isolated blood;
b) activating T cells and/or NK cells of the isolated PBMCs under effective conditions within a chamber of a closed system, comprising an effective amount of anti-CD3 antibody and/or an effective amount of anti-CD28;
c) transducing the activated T cells and/or NK cells with replication incompetent recombinant retroviral particles under effective conditions, thereby producing genetically modified T cells and/or NK cells; and
d) expanding the genetically modified T cells and/or NK cells in cell expansion media to a volume exceeding 150 ml and an expansion completion selection criteria selected from a lactate concentration exceeding 10 mM, at least a 10-fold expansion of T cells and/or NK cells, and at least 4 days in cell expansion media, wherein the activating, transducing, and expanding are performed within the chamber without washing the cells between or during the activating, transducing, and expanding.

2. The method of claim 1, wherein the expanding is performed without removing more than 10% of the cell expansion media at any time during the expanding.

3. The method of claim 1, wherein the activating, transducing, and expanding are performed in the same chamber without removing the T cells and/or NK cells from the chamber between the activating and at least 7 days of expanding the T cells and/or NK cells in cell expansion media.

4. The method of claim 1, wherein at least 5 mM more N-acetyl cysteine is present in the cell expansion media than in a transduction reaction mixture in which the transducing is performed.

5. The method of claim 1, wherein more than 1/100^{th} the effective amount of anti-CD3 antibody and/or anti-CD28 antibody are present in the cell expansion media as present in an activation reaction mixture in which the activating is performed.

6. The method of claim 1, wherein the effective conditions for the activating step comprise a concentration of isolated PBMCs between 5×10⁴ PBMCs/ml and 4×10⁶ PBMCs/ml.

7. The method of claim 1, wherein after the expansion, between 50 times and 150 times as many cells are present as the number of PBMCs that were present during the activating step.

8. The method of claim 1, wherein the activating and the expanding occur in the presence of an effective amount of IL-2.

9. A method according to claim 8, wherein the effective amount of IL-2 is between 25 IU/ml and 299 IU/ml.

10. A method according to claim 8, wherein IL-2 is present at a concentration of below 300 international units/ml for the activating and expanding.

11. A method according to claim 8, wherein IL-2 is present in the cell expansion media at the start of the expanding and is added to the cell expansion media at least two times during the expanding.

12. The method of any one of claims 8-11, wherein IL-7 is present in the cell expansion media during the expanding step.

13. The method of any one of claim 8-12, wherein the T cells and/or the NK cells are expanded at least 20-fold from the number of T cells and/or NK cells in the activation step.

14. The method of claim 1, wherein the replication incompetent recombinant retroviral particles each comprise a retroviral genome comprising one or more nucleic acid sequences operatively linked to a promoter active in T cells and/or NK cells, wherein a first nucleic acid sequence of the one or more nucleic acid sequences encodes a chimeric antigen receptor (CAR) comprising:
a) an antigen-specific targeting region (ASTR),
b) a transmembrane domain, and
c) an intracellular activating domain.

15. A method according to claim 14, wherein the ASTR is a microenvironment restricted ASTR.

16. A method according to claim 15, wherein the microenvironment restricted ASTR exhibits increased binding to its cognate antigen at a pH of 6.7 versus a pH of 7.4.

17. The method of claim 1, wherein the activating is performed in the presence of anti-CD3 antibody in solution.

18. The method of claim 1, wherein the activating is performed in the absence of anti-CD3 antibody and/or anti-CD28 attached to a synthetic solid support.

19. The method of claim 1, wherein the effective conditions for the transducing comprise incubating the activated T cell and/or NK cell in the presence of the replication incompetent recombinant retroviral particles for between 6 hours and 36 hours before adding the cell expansion media.

20. The method of claim 1, wherein the cell expansion media is a commercially available chemically-defined media for ex vivo T-cell cell expansion.

21. A method according to claim 20, wherein the cell expansion media further comprises a synthetic sera replacement.

22. A method according to claim 20 or 21, wherein the cell expansion media comprises L-glutamine or a dipeptide substitute for L-glutamine.

23. A method according to any one of claims 20-22, wherein the media has the composition of the basal media with media supplement of catalog number A1048501 or A1048503 of Thermo Fisher Scientific.

24. A method according to any one of claims 20-23, wherein the cell expansion media comprises the composition of the basal media with media supplement of catalog number A1048501 or A1048503 of Thermo Fisher Scientific, supplemented with L-glutamine or a dipeptide substitute for L-glutamine, a synthetic sera replacement, and IL-2 at a concentration of at least 50 IU/ml.

25. A method according to claim 24, wherein after the expansion, at least 25 times as many cells are present as the number of PBMCs that were present during the activating step.

26. A method according to any one of claims 20-25, wherein the cell expansion media comprises less than 300 IU/ml of IL-2.

27. A method according to claim 26, wherein the cell expansion media comprises between 50 and 150 IU/ml of IL-2 and a concentration of NAC that is at least 5 mM greater than the concentration of NAC present during the transduction reaction.

28. A method according to any one of claims 20-27, wherein natural sera is absent during expansion.

29. A method according to any one of claims 20-26 and 28, wherein the cell expansion media comprises a concentration of NAC that is between 5 mM and 20 mM greater than the concentration of NAC present during the transduction reaction.

30. The method of claim 1, wherein the effective conditions for activating do not comprise anti-CD28.

31. A method according to claim 30, wherein between 60% and 90% of the expanded cells are CD8+ T cells.

32. A method according to claim 30, wherein the expanded cells comprise at least twice as many CD8+ T cells as CD4+ T cells.

33. The method of claim 1, wherein the activation, transduction, and expansion are performed without centrifugation.

34. The method of claim 1, wherein the expanded cells comprise at least 75% T cells.

35. The method of claim 1, wherein the method is performed without enriching T cells and/or NK cells from other PBMCs before the activating step.

36. The method of claim 1, wherein the expanding is performed within a rigid cell culture container within the closed system that is permeable to gas.

37. The method of claim 1, wherein recombinant human fibronectin is not present during the activating and/or transducing.

38. The method of claim 1, wherein the activating, transducing, and expanding are performed within the same rigid cell culture container within the closed system.

39. The method of claim 38, wherein the T cells and/or NK cells are not removed from the rigid cell culture container at any point from the beginning of the activating through completion of the expanding step.

40. The method of claim 1, wherein the method further comprises harvesting the genetically modified T cells and/or NK cells after the expanding.

41. A method according to claim 40, wherein the harvesting is performed when a concentration of lactate in the cell expansion media reaches between 10 and 30 mM.

42. A method according to claim 40, wherein the harvesting is performed within 12 days of collecting the blood.

43. A method according to claim 40, wherein the harvesting is performed when the cells are expanded for between 10 and 14 days.

44. A method according to claim 40, wherein the harvesting is performed wherein no more than 10% of media is removed during performance of the method from the start of the activating until the beginning of the harvesting.

45. A method according to claim 40, wherein the cells are harvested when the transduced T cells and/or NK cells are expanded at least 10-fold.

46. A method according to any of claims 40-45, further comprising cryopreserving the harvested genetically modified T cells and/or NK cells.

47. A method according to claim 46, wherein the cryopreserved genetically modified T cells and NK cells are thawed.

48. A method according to any of claims 40-47, further comprising introducing the harvested genetically modified T cells and/or NK cells into a subject.

49. The method of claim 1, further comprising collecting blood from a subject to obtain the isolated blood.

50. A method according to claim 49, wherein the harvested genetically modified T cells and/or NK cells are reintroduced into the subject from which the blood was collected.

51. A method according to claim 50, wherein the subject is lymphodepleted at a lymphodepletion timepoint when the expanding reaches an expansion progress criteria.

52. A method according to claim 51, wherein the expansion progress criteria is selected from a lactate concentration in the cell expansion media exceeding 1 mM, at least a 2-fold expansion of T cells and/or NK cells, or a predetermined number of days of expanding.

53. A method according to claim 51, wherein the subject is lymphodepleted when a lactate concentration in the cell expansion media exceeding 5 mM.

54. The method of claim 51, wherein the subject is lymphodepleted when a lactate concentration of the cell expansion media exceeds 10 mM.

55. The method of claim 51, wherein the subject is lymphodepleted when at least a 2-fold expansion of T cells and/or NK cells is attained.

56. A method according to any one of claims 49 to 55, wherein between 50 ml and 150 ml of blood are collected.

57. A method according to claim 56, wherein the genetically modified T cells and/or NK cells are expanded to a volume between 500 ml and 2 L.

58. A method according to any one of claims 51 to 57, wherein at least 10 times as many genetically modified T cells and/or NK cells are harvested than the number of T cells and/or NK cells that were present in the isolated PBMCs.

59. The method of claim 49, wherein the subject is afflicted with cancer.

60. A method according to any one of claims 51 to 58, wherein the method is performed to treat a disease for which the subject is afflicted.

61. The method of claim 60, wherein the disease is cancer.

62. A genetically modified T cell and/or NK produced by a method according to any of the preceding claims.

63. A population of genetically modified T cells produced by a method according to any one of the preceding claims.

64. The population according to claim 63, wherein the population has a ratio of at least twice as many CD8 positive cells as CD4 positive cells.

65. A population according to any one of claims 63 to 64, wherein the cells are present in a chemically-defined media.

66. A population according to claim 65, wherein the cells are present in a media comprising recombinant IL-2.
